(19) United States
(12) Patent Application Publication
(10) Pub. No.: US 2005/0176141 A1
(43) Pub. Date: Aug. 11, 2005

(54) Compositions and methods relating to culturing neural stem cells with bone marrow stromal cells

(76) Inventors: Padmavathy Vanguri, Cockeysville, MD (US); Smita Savant-Bhonsale, Ellicott City, MD (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/048,581

(22) Filed: Feb. 1, 2005

Related U.S. Application Data
(60) Provisional application No. 60/633,521, filed on Dec. 6, 2004.
(60) Provisional application No. 60/542,581, filed on Feb. 6, 2004.

(51) Int. Cl. C12N 5/08; C12N 5/02

(52) U.S. Cl. 435/368; 435/372; 435/404

(57) ABSTRACT

The present invention encompasses methods and compositions for enhancing the growth of neural stem cells. Methods for modulating MHC molecule expression on a neural stem cell (NSC) are also included in the invention.

### CROSS-REFERENCE TO RELATED APPLICATIONS

This application claims priority pursuant to 35 U.S.C. § 119(e) to U.S. Provisional Application No. 60/633,521, filed Dec. 6, 2004 and Provisional Application No. 60/542,581, filed Feb. 6, 2004.

### BACKGROUND OF THE INVENTION

Bone marrow contains at least two types of stem cells, hematopoietic stem cells and stem cells of non-hematopoietic tissues variously referred to as mesenchymal stem cells or marrow stromal cells (MSCs) or bone marrow stromal cells (BMSCs). These terms are used synonymously throughout herein. MSCs are of interest because they are easily isolated from a small aspirate of bone marrow and they readily generate single-cell derived colonies. The single-cell derived colonies can be expanded through as many as 50 population doublings in about 10 weeks, and can differentiate into osteoblasts, adipocytes, chondrocytes (A. J. Friedenstein et al. 1970 Cell Tissue Kinet. 3:393-403; H. Castro-Malaspina et al. 1980 Blood 56:289-301; N. N. Beresford et al. 1992 J. Cell Sci. 102:341-351; D. J. Prockop 1997 Science 276:71-74), myocytes (S. Wakitani et al. 1995 Muscle Nerve 18:1417-1426), astrocytes, oligodendrocytes, and neurons (S. A. Azizi et al. 1998 Proc. Natl. Acad. Sci. USA 95:3908-3913; G. C. Kopen et al. 1999 Proc. Natl. Acad. Sci. USA 96:10711-10716; M. Chopp et al. 2000 Neuroreport II, 3001-3005; D. Woodbury et al. 2000 Neuroscience Res. 61:364-370).

Furthermore, MSCs give rise to cells of all three germ layers (Kopen, G. C. et al. 1999 Proc. Natl. Acad. Sci. 96:10711-10716; Liechty, K. W. et al. 2000 Nature Med. 6:1282-1286); Kotton, D. N. et al. 2001 Development 128:5181-5188; Toma, C. et al. 20002 Circulation 105:93-98; Jiang, Y. et al. 2002 Nature 418:41-49). In vivo evidence indicates that unfractionated bone marrow-derived cells as well as pure populations of MSCs give rise to epithelial cell-types including those of the lung (Krause, et al. 2001 Cell 105:369-377; Petersen, et al. 1999 Science 284:1168-1170) and several recent studies have shown that engraftment of MSCs is enhanced by tissue injury (Ferrari, G. et al. 1998 Science 279:1528-1530; Okamoto, R. et al. 2002 Nature Med. 8:1101-1017). For these reasons, MSCs are currently being tested for their potential use in cell and gene therapy of a number of human diseases (Horwitz et al., 1999 Nat. Med. 5:309-313; Caplan, et al. 2000 Clin. Orthoped. 379:567-570).

MSCs constitute an alternative source of pluripotent stem cells. Under physiological conditions they maintain the architecture of bone marrow and regulate hematopoiesis with the help of different cell adhesion molecules and the secretion of cytokines, respectively (Clark, B. R. & Keating, A. 1995 Ann NY Acad Sci 770:70-78). MSCs grown out of bone marrow by their selective attachment to tissue culture plastic can be efficiently expanded (Azizi, S. A., et al. 1998 Proc Natl Acad Sci USA 95:3908-3913; Colter, D. C., et al. 2000 Proc Natl Acad Sci USA 97:3213-218) and genetically manipulated (Schwarz, E. J., et al. 1999 Hum Gene Ther 10:2539-2549).

MSCs are also referred to as mesenchymal stem cells because they are capable of differentiating into multiple mesodermal tissues, including bone (Beresford, J. N., et al. 1992 J Cell Sci 102:341-351), cartilage (Lennon, D. P., et al. 1995 Exp Cell Res 219:211-222), fat (Beresford, J. N., et al. 1992 J Cell Sci 102:341-351) and muscle (Wakitani, et al. 1995 Muscle Nerve 18:1417-1426). In addition, differentiation into neuron-like cells expressing neuronal markers has been reported (Woodbury, D., et al. 2000 J Neurosci Res 61:364-370; Sanchez-Ramos, J., et al. 2000 Exp Neurol 164: 247-256; Deng, W., et al. 2001 Biochem Biophys Res Commun 282:148-152), suggesting that MSC may be capable of overcoming germ layer commitment.

Stem cells are self-renewing multipotent progenitors with the broadest developmental potential in a given tissue at a given time (Morrison et al. 1997 Cell 88:287-298). A great deal of interest has recently been generated from studies of stem cells in the nervous system, not only because of their importance for the understanding neural development but also for their therapeutic potential in the treatment of neurodegenerative diseases.

Current methods for the isolation and maintenance of embryonic and other stem cell lines depend on the use of murine embryonic fibroblasts (MEF) as a feeder layer. The frequency of embryonic stem cell clones has been noted to increase several-fold with the use of serum replacements in the culture medium (Amit et al. 2000 Dev Biol 227:271-278). The presence of basic fibroblast growth factor (bFGF) is required for the continued undifferentiated proliferation of the clonal embryonic stem cells. Current methods for the isolation, culture and expansion of human embryonic stem cells are limited by their reliance on a murine embryonic fibroblast feeder layer. It also remains to be demonstrated that a stem cell can be maintained indefinitely in an undifferentiated state in the absence of feeder cells.

To improve growth rate of human fetal brain stem cells, several different methods and growth factors have been used by a number of different groups during the last decade. It has been shown that bFGF and epidermal growth factor (EGF) are needed for expansion and maintenance of human fetal neural stem cells (hNSCs). These human NSC cultures are normally grown as free floating clusters of cells (neurospheres), but the neurospheres can not proliferate indefinitely in the presence of bFGF and EGF alone. Leukemia Inhibitory Factor (LIF) was shown to increase growth rate and prolong longevity of FGF and EGF responsive NSCs (Carpenter et al., 1999 and Wright et al., 2003). In addition to regulating growth rate LIF dynamically regulates several genes including Major Histocompatibility Complex (MHC) molecules on NSCs (Wright et al., 2003).

Human fetal brain stem cells are considered as attractive candidates for stem cell transplantation for regeneration of damaged tissues. Stem cells derived from embryonic or fetal donors require allogeneic transplantation. The transplantation of cells between genetically disparate individuals invariably is associated with risk of graft rejection by the host. Nearly all cells express products of the major histocompatibility complex, MHC class I molecules. Further, many cell types can be induced to express MHC class II molecules when exposed to inflammatory cytokines. Rejection of allografts is mediated primarily by T cells of both the CD4 and CD8 subclasses (Rosenberg et al. 1992 Annu. Rev. Immunol. 10:333). Alloreactive CD4 T cells produce cytokines that exacerbate the cytolytic CD8 response to an alloantigen. Within these subclasses, competing subpopulations of cells develop after antigen stimulation that are characterized by the cytokines they produce. Th1 cells, which produce IL-2 and IFN-γ, are primarily involved in allograft rejection (Mossmann et al., 1989 Annu. Rev. Immunol. 7:145). Th2 cells, which produce IL-4 and IL-10, can down-regulate Th1 responses through IL-10 (Fiorentino et al. 1989 J. Exp. Med. 170:2081). Indeed, much effort has been expended to divert undesirable Th1 responses toward the Th2 pathway. Undesirable alloreactive T cell responses in patients against a transplant are typically treated with immunosuppressive drugs such as prednisone, azathioprine, and cyclosporine A. Unfortunately, these drugs generally need to be administered for the life of the patient and they have a multitude of dangerous side effects including generalized immunosuppression.

Neural stem cells express low or negligible levels of MHC class I and/or class II antigens (McLaren et al. 2001 J. Neuroimmunol. 112:35), but these cells are usually rejected after implantation into allogeneic recipients unless immunosuppressive drugs are used. Rejection may be initiated after MHC molecules are up-regulated on cell membranes after exposure to inflammatory cytokines of the IFN family.

Thus, there is a strong need for standardization of culture conditions for maximizing the proliferation and multipotentiality of NSCs for therapeutic use. In addition, it is currently believed that a successful transplantation of NSCs is dependent on the prevention and/or reduction of an unwanted immune response against the NSCs mediated by immune effector cells to avert host rejection of the NSCs. Thus, there is long-felt need for methods to suppress or otherwise prevent an unwanted immune response associated with transplantation of NSCs between genetically disparate individuals. The present invention satisfies this need.

### BRIEF SUMMARY OF THE INVENTION

The invention includes compositions and methods for culturing neural stem cells (NSCs). The invention also includes a cell produced by such compositions and methods.

The invention includes a composition comprising an isolated Bone Marrow Stromal Cell (BMSC) and a chemically defined culture medium comprising Neural Stem Cell (NSC) growth medium and factors secreted by said BMSC.

In one aspect, the culture medium does not contain exogenous Leukemia Inhibitory Factor (LIF).

In another aspect, the factors secreted by the BMSCs are selected from the group consisting of growth factors, trophic factors and cytokines.

In yet another aspect, the factors are selected from the group consisting of LIF, brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-Ira, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

The invention also includes co-culturing BMSCs with NSCs in a chemically defined culture medium comprising Neural Stem Cell (NSC) growth medium.

In one aspect, the BMSCs and the NSCs are cultured in a contact dependent manner, wherein the BMSCs are physically contacted with the NSCs.

In another aspect, the BMSCs and the NSCs are cultured in a contact independent manner, wherein the BMSCs are not physically contacted with the NSCs.

In a further aspect, NSCs are derived from the central nervous system of a human.

In yet another aspect, BMSCs are derived from a human.

In another aspect, exogenous genetic material has been introduced into the cells of the present invention.

The present invention also includes a Bone Marrow Stromal Cell conditioned medium (BMSC-CM) comprising a chemically defined culture medium comprising Neural Stem Cell (NSC) growth medium and factors secreted by an isolated BMSC.

In one aspect, the BMSC-CM does not contain exogenous LIF.

In another aspect, the BMSC-CM is essentially free of BMSCs.

In yet another aspect of the present invention, the BMSC-CM comprises factors selected from the group consisting of growth factors, trophic factors and cytokines.

In a further aspect, BMSC-CM comprises factors selected from the group consisting of LIF, brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

The present invention also includes a method of modulating Major Histocompatibility Complex (MHC) molecule expression on an isolated NSC.

In one aspect, co-culturing an isolated BMSC with an isolated NSC modulates the MHC molecule expression on the NSC.

In another aspect, MHC molecule expression on an NSC can be modulated by culturing NSCs with BMSC-CM.

The present invention includes an isolated NSC prepared from co-culturing BMSCs with NSCs.

In one aspect of the invention a NSC produced by the methods of the present invention exhibits a reduced expression of MHC Class I molecule.

In another aspect, a NSC produced by the methods of the present invention exhibits a baseline level of MHC Class II molecule.

The present invention also includes a neural cell culture device comprising an isolated NSC, an isolated BMSC, a NSC growth medium, and a means for keeping the NSC and the BMSC from coming into physical contact with one another.

In another aspect, the device further comprises a filter or membrane which keeps NSCs and BMSCs from coming into physical contact with one another.

In yet another aspect, the filter or membrane has pores to allow factors secreted from said BMSC to cross said filter or membrane.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph depicting the proliferation of BMSCs in BMSC medium (DMEM-low glucose, 10% lot tested fetal bovine serum (FBS)), or NSC-medium (DMEM-F12, N2-Supplement, EGF 20 ng/ml, bFGF 10 ng/ml, Heparin 8 μg/ml, Penicillin-Streptomycin (P/S)).

FIG. 2, comprising FIG. 2A through 2C, is a series of images depicting NSCs grown as neurospheres. FIGS. 2B and 2C depict NSCs grown in the presence of exogenous leukemia inhibitory factor (LIF). FIG. 2A depicts neurospheres grown in the absence of exogenous LIF.

FIG. 3, comprising FIG. 3A through 3D, is a series of images depicting co-cultures of BMSCs and NSCs in the presence of exogenous LIF (FIGS. 3C and 3D) and in the absence of exogenous LIF (FIGS. 3A and 3B).

FIG. 4, comprising FIG. 4A through 4H, is a series of images depicting nestin and glial fibrillary acidic protein (GFAP) expression by NSCs in co-culture with BMSCs, pre-differentiation (FIG. 4A-4F). FIGS. 4G and 4H depict absence of nestin and GFAP expression by BMSCs cultured alone.

FIG. 5, comprising FIG. 5A through 5D, is a series of images demonstrating that NSCs grown on BMSCs retain their potential to differentiate into neurons and astrocytes. FIGS. 5A through 5D depict MAP2-GFAP-DAPI staining of differentiated co-cultures. MAP2 is a neuronal cytoskeletal protein. DAPI is 4′,6′-diamidino-2-phenylindole hydrochloride which stains for the nucleus.

FIG. 6 is an image depicting nestin-GFAP staining of differentiated co-cultures showing negligible nestin expression after differentiation.

FIG. 7 is an image depicting MAP2-GFAP staining of differentiated NSCs.

FIG. 8, comprising FIGS. 8A and 8B, is a series of images depicting MAP2-GFAP-DAPI staining of differentiated BMSCs showing negligible expression of neuronal and astrocyte markers.

FIG. 9, comprising FIGS. 9A and 9B is a series of FACS analysis graphs depicting the phenotype of the NSCs after BMSC depletion. FIG. 9A demonstrates that less than 2% of cells were CD-105 positive (marker for BMSC). FIG. 9B demonstrates that more than 90% of the cells were CD-133 positive.

FIG. 10, comprising FIG. 10A and FIG. 10B, is a series of FACS analysis graphs depicting the absence of nestin expression by BMSCs (FIG. 10A) and expression of Nestin by NSCs isolated from co-cultures with BMSC feeders (FIG. 10B).

FIG. 11 is an image demonstrating that NSCs grown on BMSCs retain their multipotentiality to differentiate into neurons and astrocytes.

FIG. 12 is a graph depicting growth of NSCs in Transwell™ in the presence or absence of BMSCs.

FIG. 13, comprising FIG. 13A and FIG. 13B, is a series of images depicting NSCs grown on un-coated plates in the presence of BMSCs in a Transwell™ (FIG. 13A) and absence of BMSCs (FIG. 13B).

FIG. 14, comprising FIG. 14A through 14D, is a series of images depicting NSCs cultured with bone marrow stromal cell conditioned medium (BMSC-CM) (FIGS. 14A and 14B) and NSCs cultured in NSC-medium in the presence of exogenous LIF (FIG. 14C) and absence of exogenous LIF (FIG. 14D).

FIG. 15 is a graph depicting the effects of BMSC-CM compared to standard NSC medium with or without exogenous LIF on the growth of NSCs. BMSC-CM1 is medium from BMSCs cultured in NSC-medium in the presence of EGF and FGF. BMSC-CM2 is medium from BMSCs cultured in NSC-medium in the absence of EGF and FGF.

FIG. 16, comprising FIG. 16A through 16D, is a series of FACS analysis graphs depicting the phenotypic profile of NSCs isolated from co-culture with 2 different donors of BMSCs (FIGS. 16A and 16B). FIG. 16C and FIG. 16D is a series of graphs depicting the phenotypic profile of NSCs cultured without BMSCs in NSC-medium, or NSC medium in the presence of exogenous LIF, respectively.

FIG. 17, comprising FIG. 17A through 17D, is a series of FACS analysis graphs depicting the phenotype of NSCs following culturing in NSC media in the presence and absence of exogenous LIF, and the phenotype of the NSCs following culturing in BMSC-CM in the presence and absence of growth factors. FIGS. 17A through 17D depict the profile of CD56, CD133, MHC Class II molecules and MHC Class I molecules, respectively.

FIG. 18, comprising FIG. 18A through 18D, is a series of FACS analysis graphs depicting the phenotype of NSCs following culturing in NSC medium in the presence (FIG. 18B) and absence of exogenous LIF (FIG. 18A), and the phenotype of NSC following co-culturing with BMSCs in complete NSC medium (FIGS. 18C and 18D). FIG. 18 depicts the expression of MHC Class I and Class II molecules by the NSCs cultured under the various conditions (Black=isotype control; Gray=Class I or Class II).

### DETAILED DESCRIPTION

The present invention comprises compositions and methods for inducing and/or enhancing proliferation of neural stem cells (NSCs) while preserving their multipotentiality. Also encompassed in the present invention are compositions and methods for modulating expression of MHC molecules by NSCs.

The present invention relates to the discovery that bone marrow stromal cells (BMSCs) can serve as a feeder layer to support proliferation of NSCs. As such, the present invention comprises compositions and methods for inducing and/or enhancing proliferation of NSCs while preserving their multipotentiality using BMSCs as a feeder layer to culture NSCs.

In addition, the present disclosure demonstrates that co-culturing NSCs on a BMSC feeder layer reduces the upregulation and/or induction of MHC molecule expression by NSCs as compared with the expression of MHC molecules by an otherwise identical NSC cultured in the absence of a BMSC feeder layer in neural stem cell medium (NSC-medium) supplemented with exogenous LIF for increased expansion. Thus, the present invention comprises compositions and methods for reducing and/or preventing the expression of MHC molecules by NSCs using BMSCs as a feeder layer to culture and expand the NSCs.

The data disclosed herein also demonstrate that BMSCs secrete among others, growth factors, trophic factors, and/or cytokines useful for the proliferation of NSCs while preserving their multipotentiality. The factors secreted by the BMSCs can be collected by way of culturing the BMSCs in a medium for a period of time and harvesting the conditioned medium for use at a later time. Therefore, the present invention also comprises a bone marrow stromal cell conditioned medium (BMSC-CM) useful for the proliferation of NSCs while preserving their multipotentiality.

The present invention also relates to the discovery that the factors secreted from BMSCs that are present in BMSC-CM reduces the upregulation and/or induction of MHC molecule expression by NSCs. As such, the present invention comprises compositions and methods for expanding NSCs using BMSC-CM in the absence and/or reduction of MHC molecule upregulation on the NSCs.

Accordingly, the present invention encompasses methods and compositions for generating NSCs useful for therapeutic use. Thus, the present invention comprises compositions and methods for generating NSCs useful for treating patients affected by a disease, disorder, or condition of the central nervous system. The method comprises the steps of culturing and expanding NSCs and administering the NSCs into the patient.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, the term “autologous” is meant to refer to any material derived from the same individual to which it is re-introduced.

As used herein, the term “allogeneic” refers to any material derived from a different animal of the same species.

As used herein, the term “bone marrow stromal cells,” “stromal cells,” “mesenchymal stem cells” or “MSCs” are used interchangeably and refer to the small fraction of cells in bone marrow which can serve as stem cell-like precursors to osteocytes, chondrocytes and adipocytes, and which are isolated from bone marrow by their ability to adhere to plastic dishes. Marrow stromal cells may be derived from any animal. In some embodiments, stromal cells are derived from primates, preferably humans.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiated associated proteins in a given cell. For example, expression of myelin proteins and formation of myelin sheath in glial cell is a typical example of terminally differentiated glial cell. When a cell is said to be “differentiated,” as that term is used herein, the cell is in the process of being differentiated.

“Differentiation medium” is used herein to refer to a cell growth medium comprising an additive or a lack of an additive such that a stem cell, embryonic stem cell, ES-like cell, neurosphere, NSC or other such progenitor cell, that is not fully differentiated when incubated in the medium, develops into a cell with some or all of the characteristics of a differentiated cell.

“Expandability” is used herein to refer to the capacity of a cell to proliferate, for example, to expand in number or in the case of a cell population to undergo population doublings.

“Feeder Layer” is intended to mean cells that produce growth factors, cytokines, other cell-derived products and physical support through contact necessary in co-culture to enhance proliferation and maintain undifferentiated multipotential stem cells.

“Feeder cells” is used herein to describe cells of a first tissue type that are co-cultured with cells of a second tissue type, to provide an environment in which the cells of the second tissue type can grow.

As used herein, the term “growth medium” is meant to refer to a culture medium that promotes growth of cells. A growth medium will generally contain animal serum. In some instances, the growth medium may not contain animal serum.

As used herein, the term “NSC-medium” is meant to refer to a culture medium for the culturing and expansion of NSCs. Typically, NSC-medium comprises DMEM/F12, N2 Supplement, EGF, bFGF and Heparin. In some instances, the NSC-medium may not contain growth factors (e.g., EGF and bFGF).

“Bone marrow stromal cell conditioned medium” (BMSC-CM) is used herein to refer to a medium that has been conditioned by culturing BMSCs. Based upon the present disclosure, BMSC-CM is obtained by culturing BMSCs in NSC-medium whereby the medium has been conditioned by BMSCs in culture by way of having the BMSCs secrete growth factors, trophic factors and cytokines among other compounds into the NSC-medium. BMSC-CM can be used to culture NSCs to enhance proliferation of NSCs while maintaining the multipotential capacities of NSCs. In addition, BMSC-CM can be used to culture NSCs in a manner in which MHC molecule expression can be modulated.

“Leukemia Inhibitory Factor” (LIF) is used herein to refer to a 22 kDa protein member of the interleukin-6 cytokine family that has numerous biological functions. LIF has been demonstrated to have the capacity to induce terminal differentiation in leukemic cells, induce hematopoietic differentiation in normal and myeloid leukemia cells, and stimulate acute-phase protein synthesis in hepatocytes. LIF has also been shown herein to enhance proliferation of NSCs in an undifferentiated state while maintaining the multipotentiality of the NSCs.

“Exogenous LIF” refers to LIF introduced from or produced outside an organism, cell, or system.

As used herein, the term “multipotential” or “multipotentiality” is meant to refer to the capability of a stem cell of the central nervous system to differentiate into more than one type of cell. For example, a multipotential stem cell of the central nervous system is capable of differentiating into but not limited to neurons, astrocytes and oligodendrocytes.

“Neurosphere” is used herein to refer to a neural stem cell/progenitor cell wherein nestin expression can be detected, including, inter alia, by immunostaining to detect nestin protein in the cell. Neurospheres are aggregates of proliferating neural stem cells and the formation of neurosphere is a characteristic feature of neural stem cells in in vitro culture.

“Neural stem cell” is used herein to refer to an undifferentiated, multipotent, self-renewing neural cell. A neural stem cell is a clonogenic multipotent stem cell which is able to divide and, under appropriate conditions, has self-renewal capability and can terminally differentiate into neurons, astrocytes, and oligodendrocytes. Hence, the neural stem cell is “multipotent” because stem cell progeny have multiple differentiation pathways. A neural stem cell is capable of self maintenance, meaning that with each cell division, one daughter cell will also be, on average, a stem cell.

“Neural cell” is used herein to refer to a cell that exhibits a morphology, a function, and a phenotypic characteristic similar to that of glial cells and neurons derived from the central nervous system and/or the peripheral nervous system.

“Neuron-like cell” is used herein to refer to a cell that exhibits a morphology similar to that of a neuron and detectably expresses a neuron-specific marker, such as, but not limited to, MAP2, neurofilament 200 kDa, neurofilament-L, neurofilament-M, synaptophysin, β-tubulin III (TUJ1), Tau, NeuN, a neurofilament protein, and a synaptic protein.

“Astrocyte-like cell” is used herein to refer to a cell that exhibits a phenotype similar to that of an astrocyte and which expresses the astrocyte-specific marker, such as, but not limited to, GFAP.

“Oligodendrocyte-like cell” is used herein to refer to a cell that exhibits a phenotype similar to that of an oligodendrocyte and which expresses the oligodendrocyte-specific marker, such as, but not limited to, O-4.

“Progression of or through the cell cycle” is used herein to refer to the process by which a cell prepares for and/or enters mitosis and/or meiosis. Progression through the cell cycle includes progression through the G1 phase, the S phase, the G2 phase, and the M-phase.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

As used herein, the term “exogenous” refers to any material introduced from or produced outside an organism, cell, or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding an additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### DESCRIPTION

The present invention includes a method of enhancing the proliferation while maintaining the multipotential capacities of NSCs. The method comprises isolating NSCs using methods known in the art and co-culturing NSCs with BMSCs to enhance the proliferation of NSCs while maintaining the multipotential capacities of NSCs. The culturing of the two cell types can be in a contact-dependent manner whereby NSCs physically contact BMSCs, or in a contact-independent manner whereby NSCs do not physically contact BMSCs.

The invention relates to the discovery that the expandability of NSCs (the capacity of NSCs to replicate themselves multiple times) can be increased by co-culturing these cells with BMSCs. That is, an embodiment of the present invention relates to the discovery that BMSCs can serve as supporting cells in a co-culture system for the expansion of NSCs. One skilled in the art would be able to recognize based upon the present disclosure that BMSCs can serve as a feeder layer for NSCs and provide factors including, but not limited to growth factors, trophic factors and cytokines to support the culturing of NSCs while maintaining the multipotentiality of the NSCs. The BMSC feeder layer can also serve as a monolayer on which NSCs can grow.

One skilled in the art would recognize based upon the present disclosure that BMSCs and NSCs can also be co-cultured in the presence of other growth factors known in the art to enhance proliferation of NSCs.

In the present invention, BMSCs and NSCs can be co-cultured in the absence of exogenous LIF to enhance the proliferation of NSCs while maintaining the multipotential capacities of NSCs. The present disclosure demonstrates that NSCs proliferated and expanded at levels that were significantly higher than the expansion level of NSCs cultured alone on coated plates (i.e. polyornithine/fibronection coated plates) in the presence of exogenous LIF. Thus, the present invention provides a method of culturing NSCs without the requirement of using coated plates and/or exogenous LIF.

A further embodiment of the present invention encompasses a method of depleting or separating BMSCs from NSCs in a co-culture of BMSCs and NSCs. The invention relates to the discovery that BMSCs can be depleted from such a co-culture by incubating an antibody that binds to BMSCs within the co-culture followed by a separation step including but not limited to magnetic separation. An example of an antibody that binds BMSCs is the anti-CD13 antibody. The process of magnetic separation is accomplished by using magnetic beads, including but not limited to Dynabeads® (Dynal Biotech, Brown Deer, Wis.). Further to the use of Dynabeads®, MACS separation reagents (Miltenyi Biotec, Auburn, Calif.) can be used to deplete BMSCs from the co-culture. As a result of the separation step, a population of purified NSCs can be obtained. FACS may also be used to deplete BMSCs, or alternatively, to positively select NSCs.

In the NSC culture/expansion method of the invention, the NSCs retain their multipotentiality (their capacity to differentiate into one of various cell types, such as neurons, astrocytes, oligodendrocytes and the like). In a further embodiment of the present invention, NSCs expanded using the methods of the present invention retain their ability to differentiate to a greater extent (i.e., in greater proportion) than do NSCs expanded or cultured using prior art methods.

The NSC culture/expansion methods described herein solve an essential problem in the use of NSCs for treatment of human diseases. That is, prior to the disclosure provided herein, NSCs were difficult to isolate and expand in culture (i.e., it was difficult to induce them to proliferate in sufficient number). The disclosure provided herein demonstrate that NSCs can be grown and isolated in large numbers for therapeutic uses.

The present invention also relates to the discovery that the expression of MHC molecules by NSCs can be modulated by co-culturing BMSCs with NSCs. The disclosure presented herein demonstrates that in addition to enhancing the proliferation of NSCs while preserving their multipotential capacities, co-culturing BMSCs with NSCs also reduces the upregulation and/or induction of MHC molecule expression by NSCs compared with the expression of MHC molecules by NSCs cultured using standard methods known in the art. That is, the present invention provides a method of culturing NSCs in a manner that provides additional benefits over the standard methods used for enhancing proliferation of NSCs in culture.

The co-culture system provides a method of culturing NSCs in a manner that provides benefits that are significantly better than culturing NSCs alone on a coated plate or on a coated plate in the presence of exogenous LIF. As discussed elsewhere herein, the co-culture system provides for the first time a method of producing a large number of NSCs in a manner that does not require using coated plates or exogenous LIF. Further, the co-culture system provides a method of affording additional benefits and/or benefits at a higher level compared to prior art methods of culturing NSCs alone using standard NSC-medium. These benefits include, but are not limited to enhancing the proliferation of the NSCs while maintaining the multipotential capacities of the NSCs and modulating MHC molecule expression by the NSCs.

In yet another embodiment of the present invention, NSCs can be co-cultured with BMSCs in the absence of exogenous LIF to reduce the upregulation and/or induction of MHC molecule expression by NSCs. That is, the present invention provides a method for culturing NSCs without the requirement of using exogenous LIF. The disclosure herein demonstrates that BMSCs in the co-culture system can serve as a feeder layer that provides factors including, but not limited to growth factors, trophic factors and cytokines, to the co-cultured NSCs. It is believed that the factors supplied by the BMSCs provide beneficial effects to the co-cultured NSC at a level over and above the benefits of culturing NSCs alone on coated plates with NSC-medium in the presence of exogenous LIF with respect to the expansion of the NSCs and the expression of MHC molecules by the NSCs.

The discovery that MHC molecule expression can be modulated using the methods disclosed herein provides a method of generating a population of NSCs that is useful for therapeutic, diagnostic, experimental uses and the like. For example, the decreased expression of MHC molecules by NSCs using the methods disclosed herein compared with methods known in the art provides a method of decreasing the immunogenicity of the NSCs. Preferably, the decreased expression of MHC molecules by the NSCs provides a method of increasing the success for transplantation of the NSCs into a recipient.

Co-culturing NSCs with BMSCs provides a method of modulating MHC molecule expression by the NSCs in a contact-dependent or independent manner with respect to the two cell types. In an embodiment of the present invention, NSCs can be co-cultured with BMSCs in a contact-dependent manner. Not wishing to be bound to any particular theory, the physical interaction between BMSCs and NSCs triggers beneficial effects for the proliferation and expansion of NSCs. The physical interaction between the two cell types also contributes to the modulation of MHC molecule expression by the NSCs. Preferably, co-culturing NSCs with BMSCs in a contact-dependent manner with respect to the two cell types reduces the upregulation and/or induction of MHC molecule expression by NSCs compared with the expression of MHC molecules by otherwise identical NSCs that are cultured in the absence of BMSCs on coated plates using standard NSC-medium supplemented with exogenous LIF.

In yet another embodiment of the present invention, NSCs can be co-cultured with BMSCs in a contact-independent manner to modulate MHC molecule expression by the NSCs. The present invention relates to the discovery that NSCs and BMSCs can be co-cultured in a Costar Transwell™, whereby a permeable membrane filter separates the two cell types and prevents the NSCs and BMSCs from physically contacting one another. The permeable membrane filter allows factors secreted by the BMSCs to pass through the membrane and thereby be available to contribute to the phenotype of the NSCs, for example enhancing the proliferation of the NSCs while maintaining the multipotential capacities of the NSCs and modulating MHC molecule expression by the NSCs.

As demonstrated by the experiments using Transwell™, the present disclosure indicates that BMSCs are able to support the growth and expansion of NSCs in a co-culture system in the absence of direct contact between the two cell types by way of having the BMSCs supplement the culture medium with factors secreted by the BMSCs into the culture medium. Without wishing to be bound to any particular theory, the factors secreted by the BMSCs contribute to the phenotype of the NSCs. Thus, the present invention provides a method of co-culturing BMSCs with NSCs without the requirement of having the BMSCs being a feeder layer on which NSCs are directly grown. The present invention provides a method of co-culturing BMSCs and NSCs in a contact-independent manner whereby NSCs receive benefits from BMSCs in the co-culture system by way of receiving factors secreted from the BMSCs.

One skilled in the art based upon the present disclosure would recognize that any method of keeping NSCs and BMSCs from coming into physical contact with each other can be used for the co-culture system. For example, any system/device can be used other than Transwell™ to have the cells co-cultured in a contact independent manner. Such systems/devices include, but are not limited to filters and membranes having a pore size that would prevent direct contact between the two cell types, but would allow factors to cross the filter/membrane. A benefit of using such a system/device for co-culturing BMSCs and NSCs enables a method of culturing NSCs in a system that has a continuous source of factors for the expansion of NSCs. As such, the present invention includes a composition comprising a neural stem cell culture device comprising an isolated NSC, an isolated BMSC, a NSC growth medium and factors secreted by said isolated BMSC; and a means for keeping NSCs and BMSCs from coming into contact with each other.

Based on the discovery that BMSCs can support growth of NSCs in a co-culture system in a contact-independent manner by way of providing factors secreted from the BMSCs into the culture medium, it was evaluated whether a medium conditioned by BMSCs could support the growth of NSCs in a manner that enhances the proliferation of the NSCs while maintaining the multipotential capacities of the NSCs and modulates MHC molecule expression by the NSCs. The present disclosure demonstrates that a medium conditioned by BMSCs was able to support the growth of NSCs and had a significant beneficial effect, albeit less efficient than contact dependent co-culture. Thus, the present invention provides a method of using a bone marrow stromal cell conditioned medium (BMSC-CM) for culturing NSCs without using a co-culture system. The use of BMSC-CM to culture NSCs also provides another method of generating a population of NSCs having properties equivalent to a population of NSCs cultured using the co-culture system.

In addition, the present disclosure demonstrates that BMSC-CM can substitute for the use of NSC-medium supplemented with exogenous LIF for culturing NSCs. The present disclosure demonstrates that the number of cells generated following culturing NSCs in BMSC-CM was comparable to the number of cells generated using NSC-medium supplemented with exogenous LIF. Thus, the present invention provides a method of using BMSC-CM as a source of factors beneficial for inducing proliferation of NSCs at rates equal to or higher than that of using NSC-medium supplemented with exogenous LIF.

It was also demonstrated that BMSC-CM can substitute for the use of coated plates, for example polyomithine/fibronection coated plates, for the culturing and expansion of NSCs using NSC-medium. The disclosure herein demonstrates that NSCs were able to be expanded using BMSC-CM on uncoated plates. It was observed that the expansion of NSCs using BMSC-CM on uncoated plates was at least equivalent to or greater than the expansion of NSCs cultured alone on coated plates in NSC-medium even in the presence of exogenous LIF. Thus, the present invention includes a method of culturing NSCs using BMSC-CM without the requirement of coated plates and exogenous LIF.

The use of BMSC-CM also provides a method of culturing NSCs in a manner that affords benefits to the NSCs without the use of BMSCs in a co-culture system. The present disclosure demonstrates that BMSC-CM can support the culturing of NSCs and provides benefits to NSCs equivalent to that observed when using the co-culture system comprising BMSCs and NSCs. As such, the present invention includes a method of culturing NSCs using BMSC-CM to enhance the proliferation of the NSCs while maintaining the multipotential capacities of the NSCs and modulating MHC molecule expression by the NSCs.

As demonstrated herein, BMSCs can be used to generate bone marrow stromal cell conditioned medium (BMSC-CM). BMSC-CM is a medium that has been conditioned by BMSCs in culture by way of culturing BMSCs in NSC-medium and having the BMSCs secrete among others, growth factors, trophic factors, and/or cytokines into the NSC-medium. BMSC-CM comprises growth factors, trophic factors, and/or cytokines secreted from BMSCs, and includes but is not limited to, LIF, brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-Ira, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

BMSC-CM is useful for the proliferation and expansion of NSCs while maintaining the multipotential capacity of NSCs. The use of BMSC-CM provides a method of introducing among others, growth factors, trophic factors, and/or cytokines secreted by BMSCs to NSCs for the proliferation and expansion of NSCs. The use of BMSC-CM provides a method of inducing proliferation of NSCs at rates equal to or higher than that when using NSC-medium supplemented with exogenous LIF even on uncoated surfaces. Thus, the present invention provides compositions and methods for generating large numbers of NSCs for therapeutic uses using BMSC-CM.

In addition to the use of BMSC-CM to support proliferation of NSCs while maintaining their multipotentiality, the present disclosure also demonstrates that culturing NSCs in BMSC-CM provides a method of modulating expression of MHC molecules by NSCs. Preferably, culturing NSCs in BMSC-CM reduces the expression of MHC molecules by NSCs as compared with the expression of MHC molecules by an otherwise identical NSC that was cultured in NSC medium in the presence of exogenous LIF. That is, culturing NSCs in BMSC-CM reduces and/or prevents the upregulation of MHC molecule expression.

The use of BMSC-CM to modulate the expression of MHC molecules by NSCs is based on the discovery that NSCs grown in the presence of BMSC-CM did not express MHC Class II molecules under the conditions used to detect MHC II molecules and exhibited lower levels of MHC Class I molecule as compared with an otherwise identical NSC that was cultured in NSC-medium in the presence of exogenous LIF. This observation was consistent with the observation that expression of MHC molecules by NSCs was reduced following co-culturing of BMSCs with NSCs either in a contact-dependent or contact-independent manner. In any event, whether in a contact-dependent or contact-independent manner, the reduction of MHC molecule expression by NSCs using either BMSCs as a feeder layer or culturing NSCs in BMSC-CM provides a method of reducing the expression of MHC molecules by NSC. The discovery that MHC molecule expression can be modulated using the methods disclosed herein provides a method of generating a population of NSCs that is useful for therapeutic uses. The decreased expression of MHC molecules by NSCs using methods disclosed herein also provides a method of increasing the success for transplantation of NSCs into a recipient.

Based on the present disclosure, the present invention encompasses using BMSC-CM to culture and expand NSCs while reducing the expression of MHC molecules by NSC. That is, the present invention is based on the discovery that using BMSC-CM for the expansion of NSCs reduces and/or prevents the upregulation of MHC molecule expression on NSCs as compared to an otherwise identical NSC cultured in NSC medium in the presence of exogenous LIF. As such, the present invention comprises a method of generating cells useful for therapeutic use.

It has been widely established that transplantation of cells between genetically disparate individuals (allogeneic) invariably is associated with risk of graft rejection. Nearly all cells express products of the major histocompatibility complex, MHC Class I molecules. Further, many cell types can be induced to express MHC Class II molecules when exposed to inflammatory cytokines. Rejection of allografts is mediated primarily by T cells of both the CD4 and CD8 subclasses that recognize MHC Class I and II molecules. A major goal in transplantation is the permanent engraftment of the donor graft without inducing a graft rejection immune response generated by the recipient. As such, the present invention encompasses methods for reducing and/or eliminating an immune response by cells of the recipient against grafted NSCs in the recipient by culturing the NSCs prior to transplantation using methods disclosed herein, in order to reduce the expression of MHC molecules by the NSCs. Without wishing to be bound to any particular theory, a reduction in the expression of MHC molecules by NSCs using the methods disclosed herein serves to reduce the amount of MHC molecules present on the cell membrane of the NSCs thereby reducing the immunogenicity of the NSCs in the recipient.

The present invention is also useful for obtaining NSCs that express an exogenous gene, so that the NSCs can be used, for example, for cell therapy or gene therapy. That is, the present invention allows for the production of large numbers of NSCs which express an exogenous gene. The exogenous gene can, for example, be an exogenous version of an endogenous gene (i.e., a wild type version of the same gene can be used to replace a defective allele comprising a mutation). The exogenous gene could include trophic factors for CNS regeneration or a cytotoxic gene to target cancer. The exogenous gene is usually, but not necessarily, covalently linked with (i.e., “fused with”) one or more additional genes. Exemplary “additional” genes include a gene used for “positive” selection to select cells that have incorporated the exogenous gene, and a gene used for “negative” selection to select cells that have incorporated the exogenous gene into the same chromosomal locus as the endogenous gene or both.

NSCs obtained by methods of the present invention can be induced to differentiate into neurons, astrocytes, oligodendrocytes and the like by selection of culture conditions known in the art to lead to differentiation of NSCs into cells of a selected type.

NSCs cultured or expanded as described in this disclosure can be used, before or following differentiation into selected cell types, to treat a variety of disorders known in the art to be treatable using NSCs. The NSCs are useful in these treatment methods can include those that have, and those that do not have an exogenous gene inserted therein. Examples of such disorders include but not limited to brain trauma, Huntington's disease, Alzheimer's disease, Parkinson's disease, spinal cord injury, stroke, multiple sclerosis, cancer, CNS lysosomal storage diseases and head trauma.

Isolation of NSCs and BMSCs

NSCs can be obtained from the central nervous system of a mammal, preferably a human. These cells can be obtained from a variety of tissues including but not limited to, fore brain, hind brain, whole brain and spinal cord. NSCs can be isolated and cultured using the methods detailed elsewhere herein or using methods known in the art, for example using methods disclosed in U.S. Pat. No. 5,958,767 hereby incorporated herein in its entirety by reference. Other methods for the isolation of NSCs are well known in the art, and can readily be employed by the skilled artisan, including methods to be developed in the future. The present invention is in no way limited to these or any other methods, of obtaining a cell of interest.

NSCs can be isolated from many different types of tissues, for example, from donor tissue by dissociation of individual cells from the connecting extracellular matrix of the tissue or from commercial sources of NSCs. In one example, tissue from brain is removed using sterile procedures, and the cells are dissociated using any method known in the art including treatment with enzymes such as trypsin, collagenase and the like, or by using physical methods of dissociation such as mincing or treatment with a blunt instrument. Dissociation of neural cells, and other multipotent stem cells, can be carried out in a sterile tissue culture medium. Dissociated cells are centrifuged at low speed, between 200 and 2000 rpm, usually between 400 and 800 rpm, the suspension medium is aspirated, and the cells are then resuspended in culture medium.

Sources of BMSCs and methods of obtaining BMSCs from those sources have been described in the art. BMSCs can be obtained from substantially any bone marrow including, for example, bone marrow obtained by aspiration of the iliac crest of human donors. Methods for obtaining bone marrow from donors are well known in the art and are described for example, in U.S. Pat. No. 6,653,134 and International Publication No. WO 96/30031 hereby incorporated herein in their entirety. Human mesenchymal stem cells may be purchased from Cambrex, Inc., Walkersville, Md.

Use of Isolated Neural Stem Cells

Isolated neural stem cells are useful in a variety of ways. These cells can be used to reconstitute cells in a mammal whose cells have been lost through disease or injury. Genetic diseases may be treated by genetic modification of autologous or allogeneic neural stem cells to correct a genetic defect or to protect against disease. Diseases related to the lack of a particular secreted product such as a hormone, an enzyme, a growth factor, or the like may also be treated using NSCs. CNS disorders encompass numerous afflictions such as neurodegenerative diseases (e.g. Alzheimer's and Parkinson's), acute brain injury (e.g. stroke, head injury, cerebral palsy, oncologic resection, chemotherapeutic and radiation therapy supportive care) and a large number of CNS dysfunctions (e.g. depression, epilepsy, and schizophrenia). Diseases including but not limited to Alzheimer's disease, multiple sclerosis (MS), Huntington's Chorea, amyotrophic lateral sclerosis (ALS), and Parkinson's disease, have all been linked to the degeneration of neural cells in particular locations of the CNS, leading to the inability of these cells or the brain region to carry out their intended function. NSCs isolated and cultured as described herein can be used as a source of progenitor cells and for committed cells to treat these diseases. NSCs can be used as a source of trophic factors to stimulate endogenous stem cells and CNS regeneration.

The NSCs cultured as described herein may be frozen at liquid nitrogen temperatures and stored for long periods of time, being thawed and capable of being reused. The cells are usually stored in 10% DMSO and 90% NSC medium. Once thawed, the cells may be expanded using methods described elsewhere herein.

Genetic Modification

The cells of the present invention can be genetically modified by having exogenous genetic material introduced into the cells, to produce molecules such as trophic factors, growth factors, cytokines, neurotrophins, and the like, which are beneficial to the culturing of NSCs. For example, BMSCs can be genetically modified to express and secrete EGF at a higher level compared with BMSCs that have not been genetically modified to express such a factor. Without wishing to be bound to any particular theory, a BMSC that has been genetically modified to express and secrete EGF would do so at an increased level compared with an otherwise identical BMSC that has not been genetically modified to express such a factor.

A benefit of using genetically modified BMSCs in the co-culture system is to have the engineered BMSCs continuously provide exogenous factors to the co-culture system. The exogenous factor serves to provide benefits to the cultured BMSC or NSC or both. The exogenous genetic material introduced into the BMSC may also contribute to the secretion of other endogenous factors from the engineered BMSC. In addition, the genetically modified BMSC may contribute to the secretion of endogenous factors from neighboring cells. Thus, the present invention encompasses using genetically modified BMSCs to provide a continuous supply of exogenous factors to the co-culture system, and in some instances, the exogenous genetic material introduced into the BMSC contributes to the secretion of endogenous factors from the genetically modified BMSC and/or neighboring cells. In any event, the exogenous factors and/or the endogenous factors secreted from BMSCs provide beneficial factors for the culturing and expansion of NSCs.

Further, BMSCs genetically modified to express and secrete a factor, for example EGF, can also be used to generate BMSC-CM having increased levels of EGF. In addition to providing an increased level of the exogenous factor to the BMSC-CM, the genetically modified BMSC can also contribute to the secretion of endogenous factors from the engineered BMSC and/or neighboring cells. The factors include, but are not limited to, LIF, brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

A benefit of using a genetically modified BMSC for generating BMSC-CM is to increase the levels of an exogenous factor, for example EGF by way of having EGF secreted from the engineered BMSCs into the culture medium compared with a BMSC-CM generated from an otherwise identical BMSC not genetically modified. With the increased level of EGF secreted from the genetically modified cell, more EGF is present in the BMSC-CM. In addition, the increased level of EGF secreted from the engineered BMSC may contribute to the secretion of other endogenous factors from the engineered BMSC and/or neighboring cells. BMSC-CM having an increased level of EGF and/or other factors can be useful for culturing and expanding NSCs.

A benefit of using a genetically modified BMSC for generating BMSC-CM is to increase the levels of an exogenous factor, for example, EGF by way of having EGF secreted from the engineered BMSCs into the culture medium compared with a BMSC-CM generated from an otherwise identical BMSC not genetically modified. With the increased level of EGF secreted from the genetically modified cell, more EGF is present in the BMSC-CM. In addition, the increased level of EGF secreted from the engineered BMSC may contribute to the secretion of other endogenous factors from the engineered BMSC and/or neighboring cells. BMSC-CM having an increased level of EGF and/or other factors can be useful for culturing and expanding NSCs.

In another aspect, BMSCs may be genetically modified to express a gene such as HSV-Thymidine Kinase or Green Fluorescent Protein (GFP) which could be used for their removal following expansion of NSCs in BMSC co-cultures by treatment with Gancyclovir or separation on a Flow Cytometer respectively.

In addition to genetically modifying BMSCs, the present invention encompasses genetically modified NSCs. Genetically modified NSCs can be used to replace cells that are defective in an individual. The invention may also be used to express desired proteins that are secreted. That is, NSCs can be isolated, introduced with a gene for a desired protein and introduced into an individual within whom the desired protein would be produced and exert or otherwise yield a therapeutic effect. This aspect of the invention relates to gene therapy in which therapeutic proteins are administered to an individual by way of introducing a genetically modified NSC into an individual. The genetically modified NSCs are cultured using the methods disclosed herein, isolated and implanted into an individual who will benefit when the protein is expressed and secreted by the NSC in the body.

According to the present invention, gene constructs which comprise nucleotide sequences that encode heterologous proteins are introduced into the NSCs. That is, the cells are genetically altered to introduce a gene whose expression has therapeutic effect in the individual. According to some aspects of the invention, NSCs from an individual or from another individual or from a non-human animal may be genetically altered to replace a defective gene and/or to introduce a gene whose expression has therapeutic effect in the individual.

In all cases in which a gene construct is transfected into a cell, the heterologous gene is operably linked to regulatory sequences required to achieve expression of the gene in the cell. Such regulatory sequences include a promoter and a polyadenylation signal.

The gene construct is preferably provided as an expression vector that includes the coding sequence for a heterologous protein operably linked to essential regulatory sequences such that when the vector is transfected into the cell, the coding sequence will be expressed by the cell. The coding sequence is operably linked to the regulatory elements necessary for expression of that sequence in the cells. The nucleotide sequence that encodes the protein may be cDNA, genomic DNA, synthesized DNA or a hybrid thereof or an RNA molecule such as mRNA.

The gene construct includes the nucleotide sequence encoding the beneficial protein operably linked to the regulatory elements and may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

The regulatory elements for gene expression include: a promoter, an initiation codon, a stop codon, and a polyadenylation signal. It is preferred that these elements be operable in the cells of the present invention. Moreover, it is preferred that these elements be operably linked to the nucleotide sequence that encodes the protein such that the nucleotide sequence can be expressed in the cells and thus the protein can be produced. Initiation codons and stop codon are generally considered to be part of a nucleotide sequence that encodes the protein. However, it is preferred that these elements are functional in the cells. Similarly, promoters and polyadenylation signals used must be functional within the cells of the present invention. Examples of promoters useful to practice the present invention include but are not limited to promoters that are active in many cells such as the cytomegalovirus promoter, SV40 promoters and retroviral promoters. Other examples of promoters useful to practice the present invention include but are not limited to tissue-specific promoters, i.e. promoters that function in some tissues but not in others; also, promoters of genes normally expressed in the cells with or without specific or general enhancer sequences. In some embodiments, promoters are used which constitutively express genes in the cells with or without enhancer sequences. Enhancer sequences are provided in such embodiments when appropriate or desirable.

The cells of the present invention can be transfected using well known techniques readily available to those having ordinary skill in the art. Exogenous genes may be introduced into the cells by standard methods are employed for introducing gene constructs into cell which will express the proteins encoded by the genes. In some embodiments, cells are transfected by calcium phosphate precipitation transfection, DEAE dextran transfection, electroporation, microinjection, liposome-mediated transfer, chemical-mediated transfer, ligand mediated transfer or recombinant viral vector transfer.

In some embodiments, recombinant adenovirus vectors are used to introduce DNA with desired sequences into the cell. In some embodiments, recombinant retrovirus vectors are used to introduce DNA with desired sequences into the cells. In some embodiments, standard CaPO₄, DEAE dextran or lipid carrier mediated transfection techniques are employed to incorporate desired DNA into dividing cells. Standard antibiotic resistance selection techniques can be used to identify and select transfected cells. In some embodiments, DNA is introduced directly into cells by microinjection. Similarly, well-known electroporation or particle bombardment techniques can be used to introduce foreign DNA into the cells. A second gene is usually co-transfected or linked to the therapeutic gene. The second gene is frequently a selectable antibiotic-resistance gene. Transfected cells can be selected by growing the cells in an antibiotic that will kill cells that do not take up the selectable gene. In most cases where the two genes are unlinked and co-transfected, the cells that survive the antibiotic treatment have both genes in them and express both of them.

The following examples are presented in order to more fully illustrate the preferred embodiments of the invention. These examples should in no way be construed as limiting the scope of the invention, as defined by the appended claims.

### EXAMPLES

### Example 1

### BMSCs as Feeder Cells for Growth and Expansion of NSCs

Culturing of NSCs is challenging because they require growth factors and a specific substratum for enhancing their growth rate and expansion. In terms of growth factors, the addition of exogenous LIF to serum free defined growth medium containing FGF and/or EGF significantly enhances the expansion of NSCs. As discussed elsewhere herein, the combination of exogenous LIF and coating the culture dishes further increases the expansion level of the NSCs while maintaining their multipotentiality.

Bone marrow stromal cells (BMSCs) can be easily obtained and expanded in culture to a substantial homogeneous population of cells. In addition, BMSCs secrete several trophic factors that may promote NSC growth. Therefore, the present Example demonstrates that BMSCs can serve as supporting cells in a co-culture system for the expansion of NSCs.

The Materials and Methods used in the experiments presented in this Example are now described.

Establishment, Maintenance and Characterization of Human Fetal Neural Stem Cells (NSCs)

Human brain (from 11-14 week old fetuses) was received from Advanced Bioscience Resources Inc. (Alameda, Calif.). The brain tissue was triturated in cold PBS. Cells were pelleted by centrifugation and resuspended in 10 ml of NSC growth medium (DMEM/F12, 8 mM glucose, glutamine, 20 mM sodium bicarbonate, 15 mM HEPES, 8 μg/ml Heparin, N2 supplement, 10 ng/ml bFGF, 20 ng/ml EGF). The cells were plated on a T-25 cm² flask coated with polyornithine and fibronectin and grown in a 5% CO₂ incubator at 37° C. Cultures were fed by replacing 50% of the medium with fresh medium every other day and passaged by trypsinization every 14 days. Cells were cryopreserved in NSC medium with 10% DMSO in vapor phase of Liquid N₂. The cells are thawed and plated in complete growth medium supplemented with 10 ng/ml LIF after growing them initially for about 1-2 passages in the presence of bFGF and EGF.

Other NSC growth media can be used for the methods disclosed herein. For example, the cells can be cultured in Neurobasal medium supplemented with L-glutamine, bFGF, EGF, and B27 without retinoic acid (Invitrogen, Carlsbad, Calif.). Another NSC growth medium is NeuroCult supplemented with growth factors (StemCell Technologies, Vancouver BC, Canada). Without wishing to be bound to any particular theory, any media can be used to culture NSCs. However, a suitable medium allows for the cells to grow and expand while maintaining their potential to differentiate into multiple cell types.

For characterization, NSCs were plated on coated chamber slides at different passages, fixed with 4% paraformaldehyde and stained for nestin and glial fibrillary acidic protein (GFAP). NSCs were differentiated for about 14 days by withdrawing bFGF, EGF and LIF, and treating the NSCs with Neurobasal medium, B27 supplement and BDNF. Other differentiating conditions may be used to drive cells towards more specific lineages. The cells were fixed and stained for microtubule-associated protein (MAP2; a maker for neurons) and GFAP (a marker for astrocytes). To identify neuronal subtypes, the cells were stained with anti-γ-amino butyric acid (GABA), anti-tyrosine hydroxylase (TH). The primary antibodies used were human specific nestin, 1:10 (R & D Systems); MAP2, 1:500 (Sigma); GFAP, 1:1000 (DAKO); O4, 1:100; NG-2 (1:200) (Chemicon). The secondary antibodies used in these experiments were Alexa Fluor 488 chicken anti-mouse, 1:500 (Molecular Probes) and Alexa Fluor 594 chicken anti-rabbit 1:500 (Molecular Probes).

NSCs were analyzed by flow cytometry for expression of several markers including hematopoietic cell markers CD45 and CD14, stem cell markers CD34 and CD133, CD56 and Immunogenic/stimulatory markers CD80, CD86, MHC Class I and MHC Class II.

Quantitative analysis of nestin expressing cells was also determined by flow cytometry. This analysis was performed with anti-nestin (R&D) and goat anti-mouse Ig conjugated to Alexa-fluor 488. The NSCs were fixed and permeabilized using the Cytofix/Cytoperm kit and instructions from Becton-Dickinson with minor modifications.

Establishment Maintenance and Characterization of Human Bone Marrow Stromal Cells (BMSCs)

BMSCs were produced by methods know in the art. For example, human bone marrow was collected via needle aspirate. A nucleated cell count was performed on the bone marrow. Bone marrow was diluted with PBS and mixed with Hespan. The Hespan/cell suspension mix was allowed to sit undisturbed for about 45 minutes to an hour. During this period, red blood cells (RBCs) settled out which allowed for the collection of the nucleated cells present in the supernatant layer. The nucleated cells were washed and counted after debulking of the RBCs.

Nucleated cells were cultured in tissue culture treated vessels, for example polystyrene, in DMEM-low glucose, with 10% FBS. A primary culture was observed to last for about 12 to 17 days with media changes every 3 or 4 days. When there were a sufficient number of adherent, spindle shaped cells, the culture was passaged using trypsin to remove the adherent cells. Cells were re-plated and kept in culture for about one week, with one media change for every subsequent passage.

The cells were tested for purity using specific markers by flow cytometry. BMSCs of passages 1 or 2 (P1 or P2) that were CD45 negative and were greater than 90% CD90 and CD13 positive were used in the co-culture experiments.

Culture of BMSCs in NSC Growth Medium

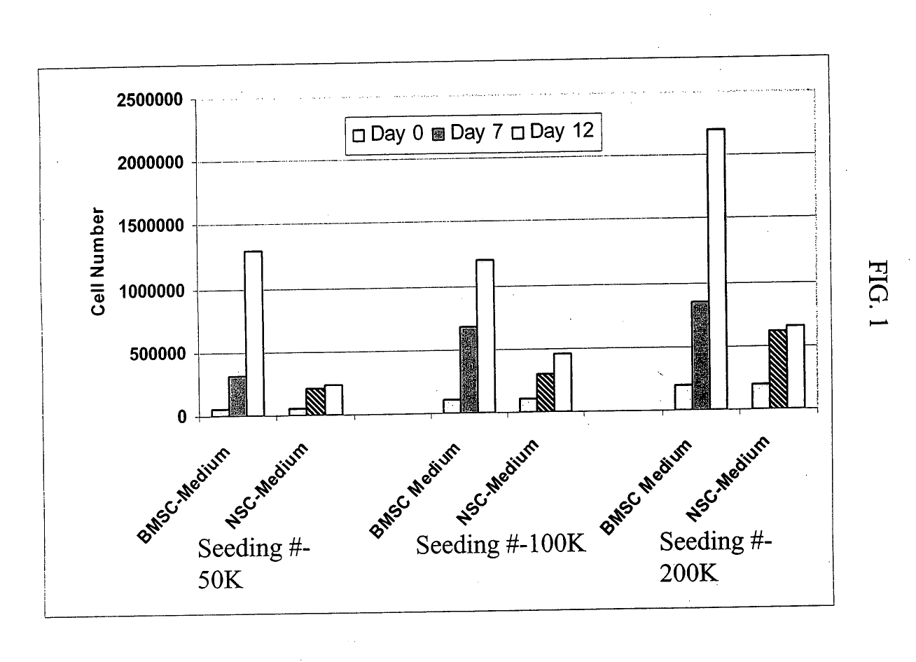

BMSCs were plated at different densities in 6-well plates and were allowed to attach overnight in BMSC medium (DMEM-low glucose, 10% Lot tested FBS). The following day, medium from one plate was replaced in NSC-medium (DMEM-F12, N2-Supplement, EGF, 20 ng/ml, bFGF, 10 ng/ml, 8 μg/ml Heparin, P/S). The other plate received fresh BMSC medium. The cells were fed with fresh BMSC or NSC-medium every 3rd day. One set of cultures was trypsinized and the cells were counted after 7 days. Another set of cells was harvested and counted after 12 days. As shown in Table 1 and FIG. 1, BMSCs proliferated much more in BMSC medium than they did in NSC-medium. In NSC-medium, the cells exhibited an initial increase in cell number that was comparatively less than that observed in BMSC medium. However, after 7 days of culture in NSC-medium, there was no significant increase in BMSC cell number. In BMSC culture medium, the cells continued to proliferate over the period of 12 days and were highly confluent at the end of this time. In NSC culture medium, the BMSCs did not form any swirls of confluent cells but appeared morphologically normal without visible cell death. This was in contrast to the morphology of the cells observed in BMSC medium.

TABLE 1

| | | Cell number | |
|---|---|---|---|
| Medium | Starting cell # | at 7 days | Cell number at 12 days |
| NSC-medium | 50,000 | 210,000 | 240,000 |
| NSC-medium | 100,000 | 300,000 | 460,000 |
| NSC-medium | 200,000 | 615,000 | 650,000 |
| BMSC-medium | 50,000 | 310,000 | 1,300,000 |
| BMSC-medium | 100,000 | 683,000 | 1,200,000 |
| BMSC-medium | 200,000 | 855,000 | 2,200,000 |

These results demonstrate that when NSCs were grown together with BMSCs in NSC-medium, the BMSCs did not compete with and outgrow the NSCs.

Co-Culture of BMSCs and NSCs; Survival and Expansion of NSCs on BMSCs vs. Coated Plates

Human BMSCs (hBM-03-016, P1) were thawed, washed, counted and plated in two 12 well plates at 75,000 cells per well. In parallel, other 12 well plates were coated with 15 μg/ml polyornithine followed by 10 μg/ml human fibronectin overnight. On the next day, hNSCs growing in culture were harvested and resuspended in NSC-medium containing EGF and FGF. The NSCs used were hHB-007, P7 which were cultured in NSC-medium containing EGF, FGF and LIF. The majority of these cells were growing as neurospheres. At the time of plating these cells on to BMSCs or coated wells, the neurospheres were broken up as much as possible into single cells. The NSCs were plated in triplicate under the following conditions either on BMSCs or on coated plates.
1. 25,000 NSCs+NSC-medium, no LIF
2. 25,000 NSCs+NSC-medium+LIF
3. 50,000 NSCs+NSC-medium, no LIF
4. 50,000 NSCs+NSC-medium+LIF

The cultures were fed every other day by replacing 50% of spent medium with fresh medium. Phase contrast pictures of the cells were taken at different times. After 12-13 days in culture, the cells were harvested using trypsin. Briefly, cells were incubated with 0.05% trypsin for 5 minutes or until all the cells lifted off the dish. Trypsin was then neutralized with soybean trypsin inhibitor (SBTI). The cells were washed with phosphate buffer saline (PBS) and then counted using trypan blue dye in a hemocytometer. Two different sized cells were seen under the microscope, the larger BMSCs and smaller NSCs. The two populations were counted separately. The first count was calculated on day 12 by pooling two of the 3 replicate cultures.

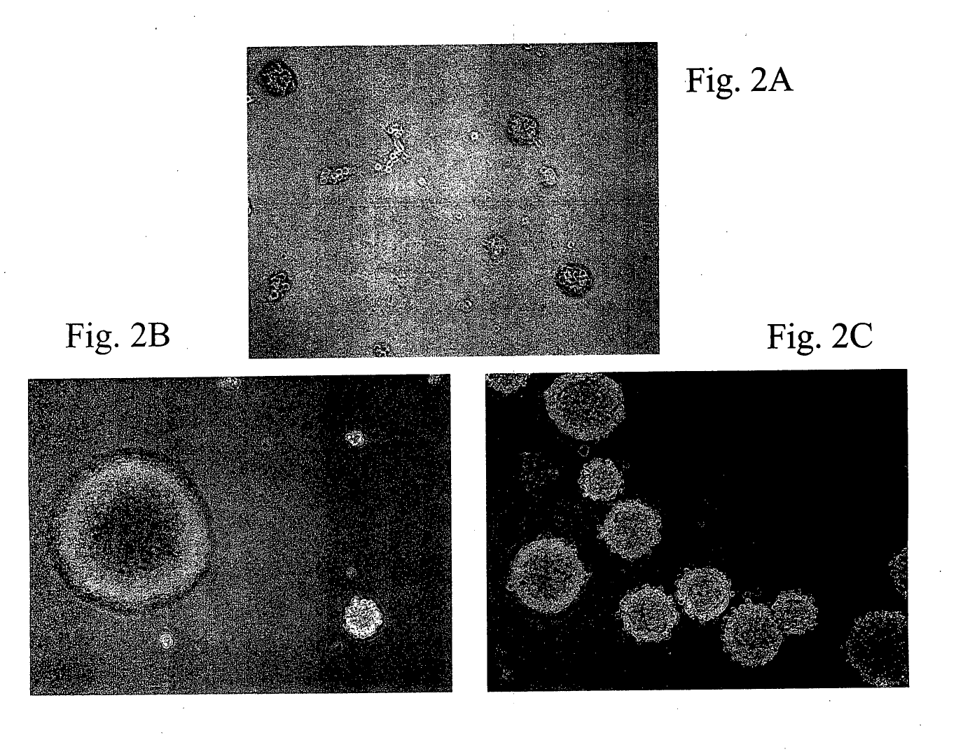

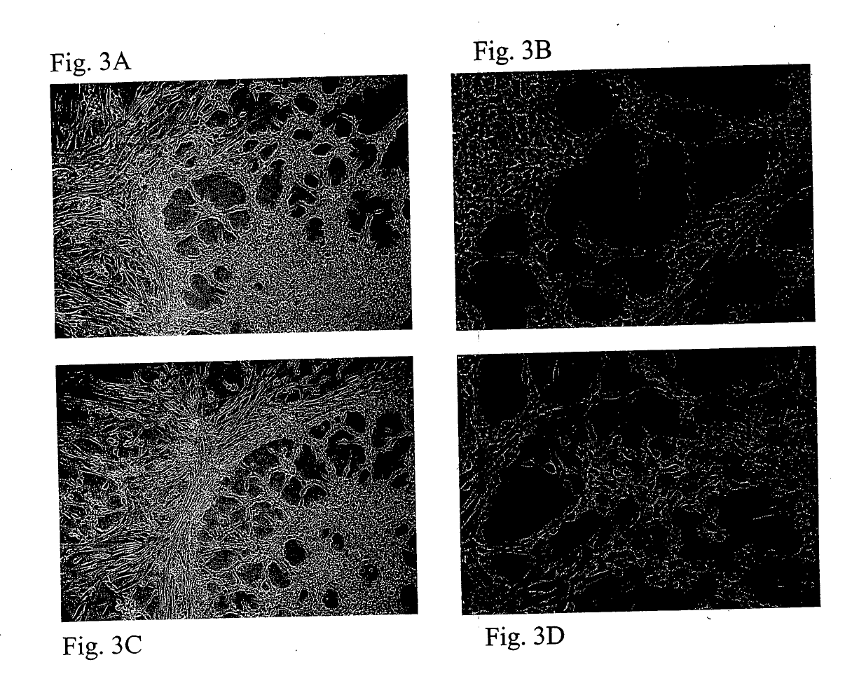

NSCs plated on BMSCs attached and spreaded out as monolayers in concentrated areas. Changing medium on these cells was not difficult. On the other hand, NSCs especially those grown in the presence of exogenous LIF as neurospheres (FIG. 2) were not very adherent, and thus extreme care had to be taken when changing the medium. Centrifugation of aspirated medium was sometimes required in order to prevent loss of the neurospheres. Also when NSCs were plated at low densities, they did not survive unless they were co-cultured with BMSCs. Morphologically the NSCs grown in co-culture formed a network of small round to oval cells on top of the flat larger BMSCs. The NSCs formed isolated colonies of NSCs, sometimes surrounded by a ring of fibroblastic BMSCs (FIG. 3). With time in culture, the NSC colonies became larger with the number of NSCs within each colony increased. NSCs grown alone survived and proliferated only in the presence of exogenous LIF and at higher seeding densities; however, in co-cultures, significant expansion of NSCs occurred even in the absence of exogenous LIF. Thus, the BMSCs appeared to serve as a feeder layer and provided trophic support to the NSCs. Significant expansion of NSCs was obtained in co-culture that was comparable to that of NSCs grown on coated dishes (i.e. polyomithine/fibronectin) with the addition of exogenous LIF. Table 2 depicts the number of NSCs and BMSCs harvested after 12 days in culture.

TABLE 2
Expansion of NSCs Grown in Co-Culture with BMSCs

| Starting # NSC | LIF | +BMSC | No BMSC |
|---|---|---|---|
| 2.5e4 | − | 8.8e4 | 1.3e4 |
| 2.5e4 | + | 10e4 | 7.5e4 |
| 5.0e4 | − | 25e4 | 11e4 |
| 5.0e4 | + | 24e4 | 33e4 |

Characterization of NSCs Cultured on BMSCs: Expression of Nestin

In order to assess nestin expression of NSCs in co-culture with BMSCs by immuno-staining, the cultures were grown on coverslips. Coverslips (10 mm) placed in 24 well dishes were coated with polyornithine followed by human Fibronectin as described elsewhere herein. One set of coverslips was plated with BMSCs, (35,000/coverslip) and the cells were allowed to attach overnight. The next day, NSCs growing in culture (THD-015+LIF, P12) were harvested and counted. Twenty five thousand NSCs/well were plated either on top of BMSCs or directly on top of the coated coverslips. Some wells with BMSCs were cultured alone without NSCs. All of the different cultures were grown either in NSC-medium or NSC-medium+LIF. The cells were fed with 50% fresh medium every other day for 10 days. After 10 days, some cultures were fixed and prepared for nestin staining. Other cultures were allowed to differentiate for two weeks.

In order to fix the cultures, the cultures were washed with PBS once. Then 4% paraformaldehyde was added and the cultures were incubated at room temperature for 15 min followed by 3 washes with PBS. At this point, the fixed cultures were stored in PBS at 4° C. or were stained right away.

Nestin staining of the following cultures was accomplished by staining with anti-nestin antibody+anti-GFAP antibody. The cultures included BMSC+NSC, BMSC+NSC+LIF, BMSC alone, BMSC+LIF, NSC alone and NSC+LIF. A duplicate sample was used as control (no primary antibodies).

NSCs cultured alone in NSC medium with or without exogenous LIF expressed nestin. When cultured in NSC medium+LIF, in addition to expressing nestin the NSCs co-expressed GFAP.

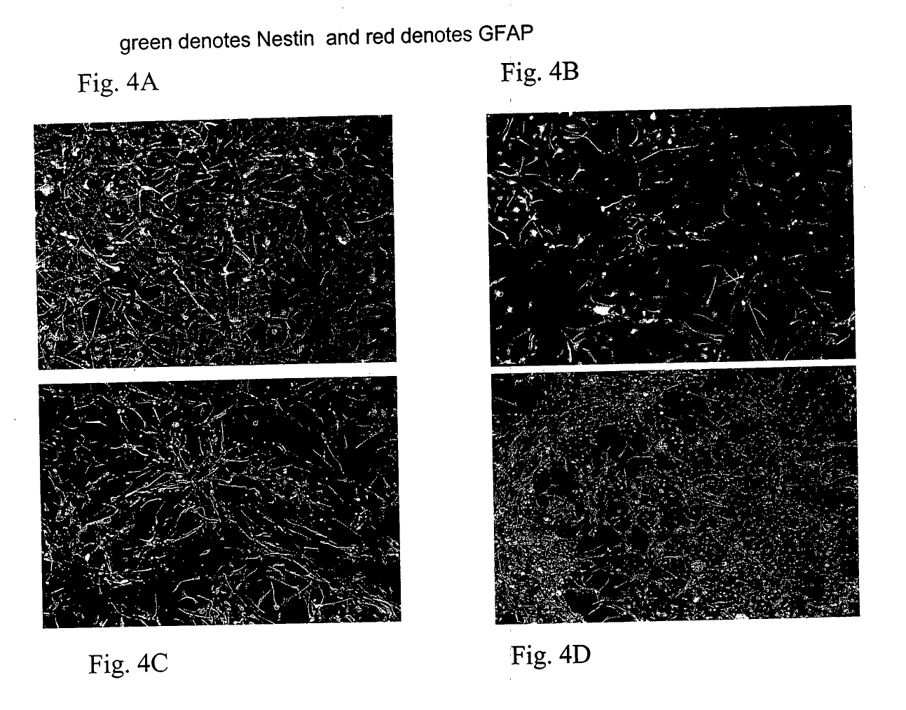

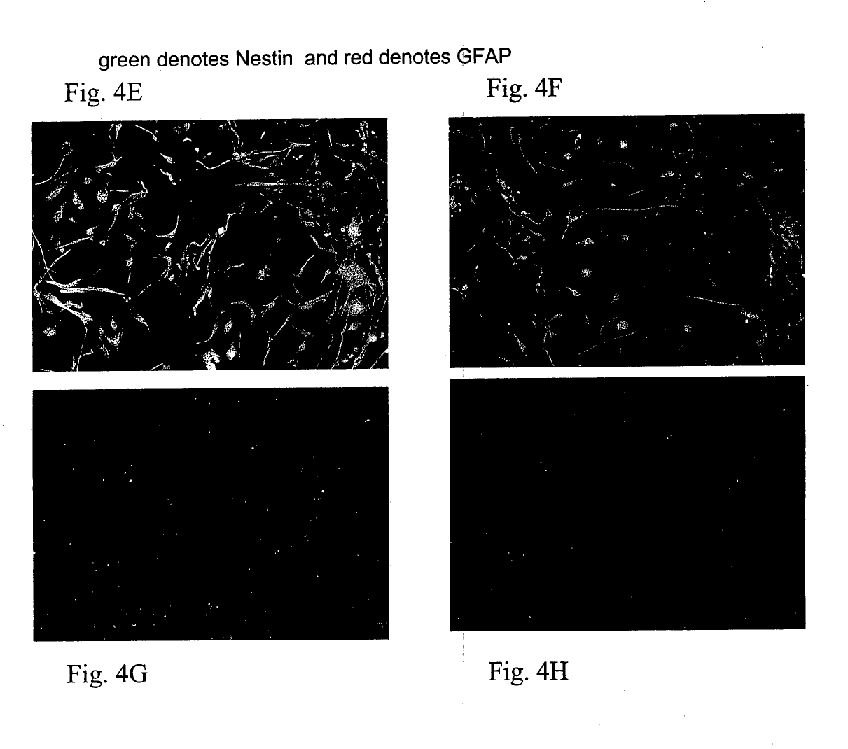

All of the co-cultures exhibited numerous nestin-positive cells spread out on top of BMSCs (FIGS. 4A-4F). The BMSCs themselves exhibited only faint background staining (FIGS. 4G-4H). The morphology of the nestin-positive cells was heterogeneous. Many of the nestin-positive cells were also positive for GFAP while others were nestin positive and GFAP negative. Some of the nestin positive cells were small, round or elliptical with or without one or two filamentous extensions. Some of the nestin-GFAP double positive cells were larger, flat and resembled astrocytes. There was no obvious difference in the co-cultures that were grown with or without the addition of exogenous LIF.

Differentiation of Co-Cultures

After growing in NSC-medium for 10 days, the co-culture was subjected to a differentiation schedule for two weeks. The differentiation schedule encompassed two feedings each of 1) DMEM/F12 with N2 supplement but no EGF or FGF, 2) DMEM/F12+B27 supplement and 3) DMEM/F12+B27+BDNF. The differentiated cultures were fixed in paraformaldehyde and then stained with the following combination of antibodies: for example nestin/GFAP, MAP2/GFAP and O4/GFAP. In all cases, the GFAP was detected with red fluorescence (Alexa 594) while nestin, MAP2 or O4 was detected with green fluorescence (Alexa 488). The cultures were counter-stained with DAPI to label all nuclei with blue fluorescence.

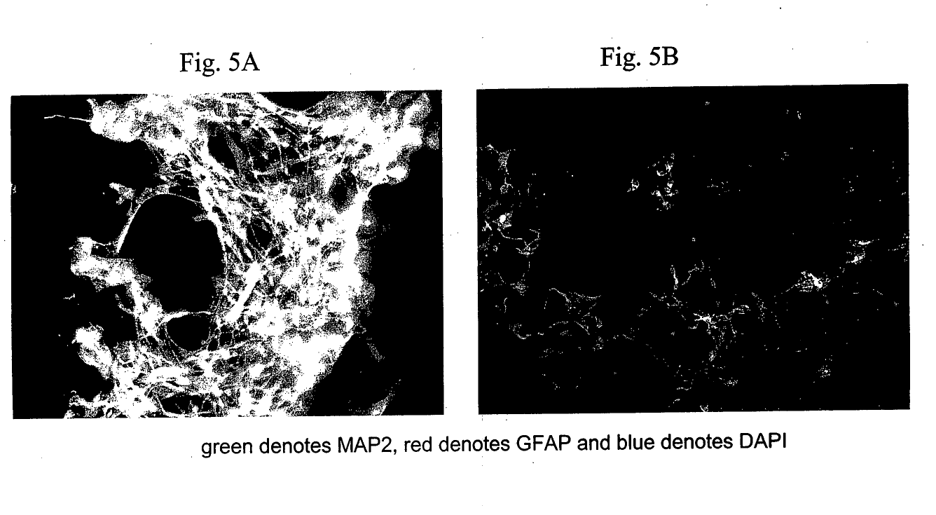

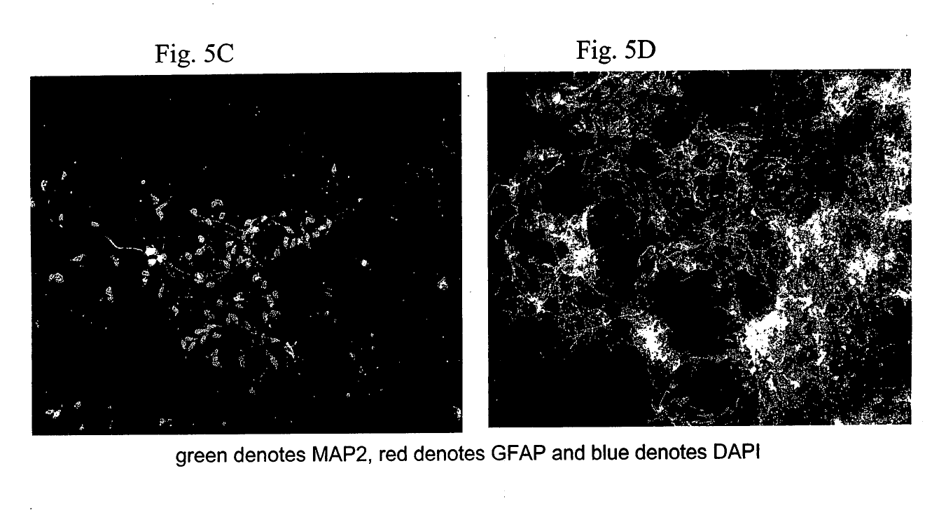

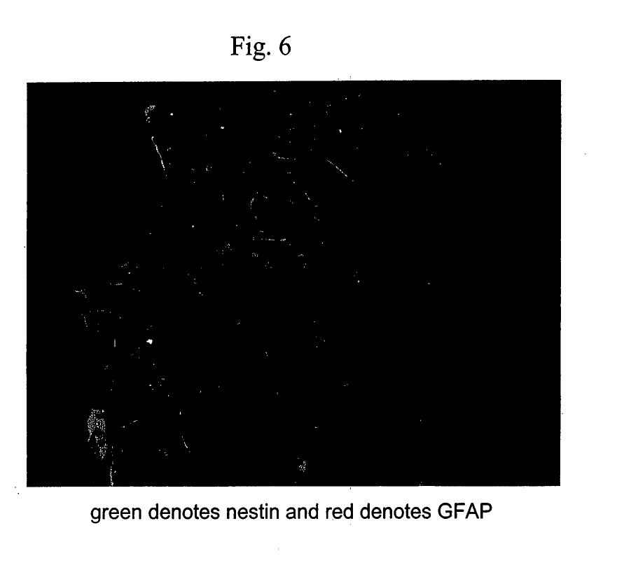

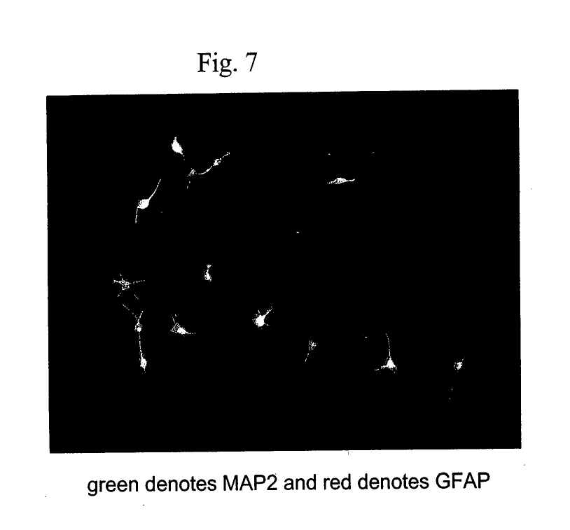

NSCs growing on BMSCs retained their potential to differentiate into neurons and astrocytes (FIGS. 5A-5D). After growing in differentiation medium, several cells expressed the neuronal marker MAP-2. These were small cells with elliptical nuclei and bipolar or multipolar cell bodies. The processes were extensive and formed intricate mesh work of neurons on top of BMSCs or on top of cells that stained for GFAP. GFAP-positive astrocytes were also present throughout the culture. These were larger, polygonal and flat when compared with the neurons. By approximation, there were either equal or more neurons than astrocytes, as a precise determination was not possible because of the extensive network of neurons forming a 3-dimensional mesh of cells. At the dilution used, it appeared that staining with O4 antibody revealed that no cells differentiated into oligodendrocytes. A longer period of differentiation may be required to generate oligodendrocytes. However, when the presence of oligodendrocytic cells were assessed using NG2 (a chondroitin sulfate proteoglycan found on surface of oligodendrocytic precursors) as a marker for oligodendrocytic differentiation, it was observed that few cells were positive for NG2. Staining of differentiated co-cultures for nestin revealed some GFAP-positive astrocytic cells that were dimly positive for nestin, but these appeared to be morphologically different from the smaller filamentous cells seen prior to differentiation (FIG. 6). The presence or absence of exogenous LIF during growth made no difference to the differentiation of NSCs in co-cultures. When the NSCs that were cultured alone were differentiated as described above, it was observed that they became neurons (MAP2 positive) and astrocytes (GFAP positive) (FIG. 7).

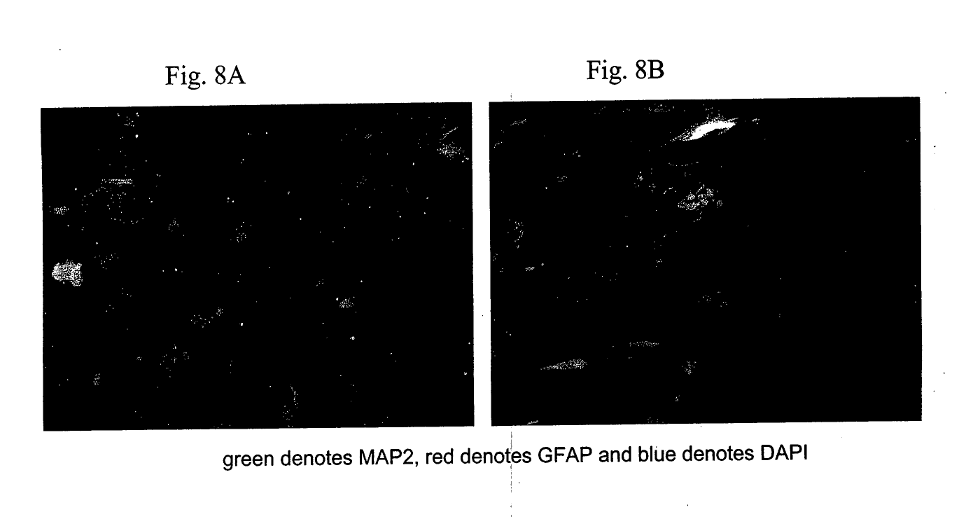

When BMSCs alone were subjected to the same differentiation conditions, they showed dim and diffuse staining for nestin and GFAP with GFAP being somewhat more intense in cells cultured with additional exogenous LIF. The morphology of the cells remained the same as BMSCs and was not astrocytic. There were rare cells in the culture that were highly nestin or MAP2 positive suggesting some differentiation into NSC or neuronal lineages (FIG. 8).

### Example 2

### Depletion of BMSCs from Co-Cultures of BMSCs and NSCs

Cells from the co-cultures of BMSCs and NSCs can be separated by incubating the co-cultures with mouse anti-human CD13 antibody (positive on BMSCs and negative on NSCs). Using magnetic beads linked to pan mouse IgG, BMSCs can be separated from NSCs. Unbound cells representing NSCs are then analyzed by FACS or are placed back into culture. For FACS analysis, a different BMSC marker antibody (mouse anti-human CD105) was used to detect contaminating BMSCs while NSCs were detected with mouse anti-human CD133.

BMSCs were plated into 6 well dishes at 150,000 cells/well and were allowed to adhere overnight in BMSC medium. NSCs, THD-hWB-015+LIF P17, growing in culture were harvested and re-suspended to break up the neurospheres. BMSC medium was removed from the BMSC cultures and the harvested NSCs (75,000 cells/well) were plated on top of the BMSCs in NSC-medium containing EGF and FGF, with or without exogenous LIF. The co-cultures were maintained for 13 days in the NSC-medium. The cells were fed every other day or over the weekend by replacing half of the spent medium with fresh medium.

On day 13, the co-cultures were harvested with trypsin followed by soybean trypsin inhibitor. The cell mixture was re-suspended in PBS containing 0.1% BSA. The cell mixture was incubated with anti-CD13 antibody for 30 minutes on ice. The cells were then washed with PBS/0.1% BSA by centrifugation. Simultaneously Dynal beads-Pan mouse IgG were washed 3× with PBS/0.1% BSA by separating them each time on a magnetic particle concentrator (Dynal-MPC). The washed cells were incubated with the washed magnetic beads in PBS/0.1% BSA at 4° C. on a tilting/rotating apparatus (Dynal Sample mixer) for 30 minutes. The tube containing the cell-bead mixture was placed in the MPC for 2-3 minutes. The beads with attached cells adhere to the side of the tube closest to the magnet. The supernatant was collected and placed into a separate tube.

A portion of the cells in the supernatant were used for FACS analysis and a portion of the cells were plated in a chamber slide coated with polyornithine and fibronectin. FACS analysis was performed with anti-CD105 (recognizes BMSCs) and anti-CD133 (recognizes NSCs). The cells plated onto chamber slide were cultured for 1-2 days in NSC-medium and then fixed for immunostaining purposes for staining with nestin/GFAP.

Over the period of two weeks, the NSCs expanded on the layer of BMSCs. Some large colonies were seen early on and presumably originated from small neurospheres. Other NSCs that began as single or a few cells grew into several smaller colonies of NSCs over the period of two weeks.

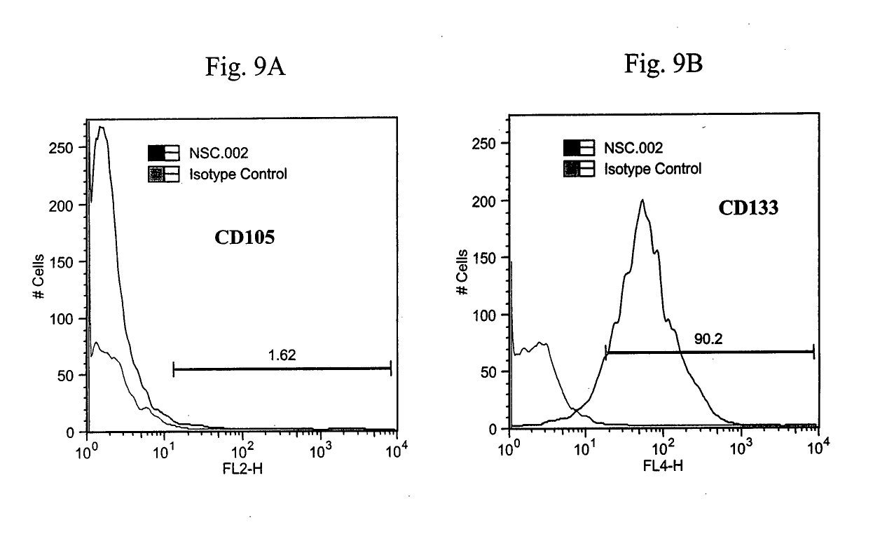

Following magnetic depletion of BMSCs, the separated NSCs were analyzed by FACS. Greater than 90% of the cells were CD1-33 positive (FIG. 9B). Less than 2% were CD105-positive (FIG. 9A). The cells that were plated on the chamber slide adhered to the dish and morphologically resembled NSCs. Immunostaining for nestin was used to verify the presence of NSCs. The BMSC-bead mixture which was placed in culture appeared morphologically like BMSCs and attached to the tissue culture flask with numerous bound magnetic beads. The results indicate that BMSCs can be successfully depleted from co-cultures by simple incubation with an antibody that binds to BMSCs followed by magnetic separation leaving an enriched population NSCs.

### Example 3

### Scale-Up of Co-Culture and Enhanced Expansion of NSCs Using Minimal BMSCs for the Feeder Layer

In this experiment the co-cultures were expanded to T-75 tissue culture flasks. BMSCs (donors BM-022, P1 and BM-024, P1) were plated at two different seeding densities of about 2.5e5 or 5.0e5 per flask in BMSC medium. Two days later, the cells were washed with NSC medium and 5.0e5 NSCs (THD-015, P14) were plated in 15 ml complete NSC medium on top of the BMSCs. The cells were co-cultured for 15 days by feeding every other day or third day by replacing half of the medium with fresh NSC medium. After 15 days, the cells were harvested by trypsinization. The larger BMSCs and smaller NSCs were counted on a hemacytometer. The BMSCs were depleted as described elsewhere herein, for example, in Example 2. The cells were re-suspended at about 5×10 e7/ml and incubated with Anti-human CD13 antibody on ice for 15 minutes. The cells were washed once and then incubated with washed Pan-mouse IgG Dyna-beads for 30 minutes. The bead-bound cells were then separated on a Magnetic Particle Concentrator. The supernatant containing the NSCs was washed with NSC medium and the number of recovered NSCs was counted. Some of the cells were analyzed by FACS, others were subjected to differentiation and immunocytochemistry.

The disclosure presented herein demonstrates that culturing NSCs on feeder layers of fewer rather than larger number of BMSCs generates greater NSC expansion. By changing the ratio of BMSC to NSC seeding densities, it was discovered that when BMSCs were plated at a low confluence of about 30%, a higher fold expansion of NSCs was observed than when BMSCs were plated at 50-60% or higher confluence. For example at seeding densities of BMSCs at around 2.5e5 and NSCs at 5.0e5 in a T-75 flask as shown in Table 3, it was observed that as much as 82 fold expansion of NSCs compared to 47 fold when a similar number of NSCs were plated on double the number of BMSCs. In comparison, the maximum expansion of NSCs attained in other experiments when NSCs were cultured in the absence of BMSCs on coated dishes in standard NSC medium supplemented with exogenous LIF was about 20-25 fold. Following the expansion of NSCs in the presence of BMSC, the BMSCs were efficiently depleted from the co-cultures using anti-BMSC antibodies and immuno-magnetic beads at 83-93% recovery rates (Table 3).

TABLE 3
Expansion of hNSCs on BMSC Feeder cells

| BMSC | | | | | # NSCs |
|---|---|---|---|---|---|
| donor | | | Fold | % Recovery | Recovered |
| (seeding | Starting # | # NSCs | Expansion | after BMSC | (Fold Ex- |
| density) | NSCs | at 15d | of NSCs | Depletion | pansion) |
| BM-03-022 | 5.0e5 | 41.1e6 | 82.2× | 93% | 38.3e6 |
| (about 2.5e5) | | | | | (76.5×) |
| BM-04-024 | 5.0e5 | 23.5e6 | 47× | 83% | 19.4e6 |
| (about 5.0e5) | | | | | (39×) |

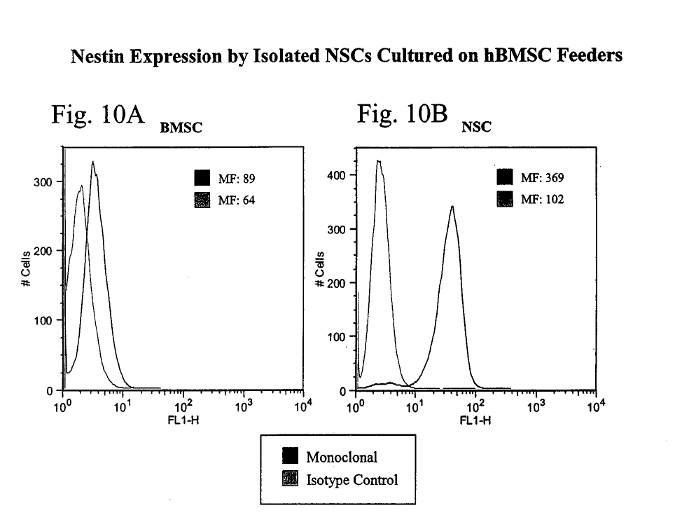

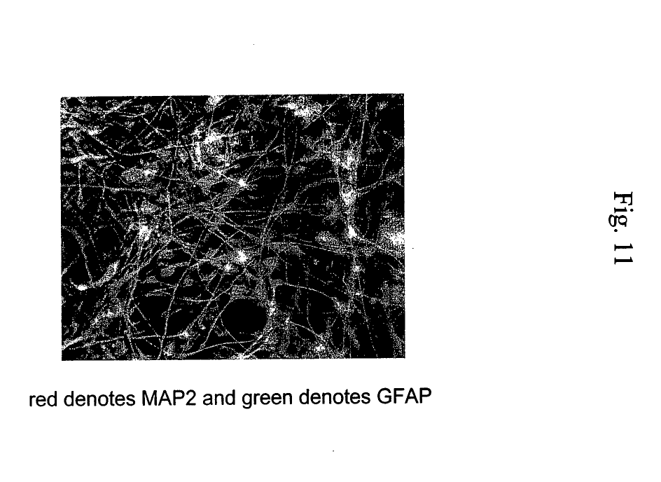

The co-culture expanded and isolated NSCs were observed to continue to express markers of progenitor cells, including but not limited to, nestin (FIG. 10). The isolated NSCs also retained their potential to differentiate into neurons and astrocytes (FIG. 11). One skilled in the art based upon the present disclosure would be able to recognize that this method for expanding NSCs could be further scaled up and adapted to a closed system manufacturing process that has been validated for the production of BMSCs.

### Example 4

### Co-Culture of BMSCs and NSCs in Transwells™ (Contact-Independent Co-Culture)

It has been demonstrated herein that BMSCs are capable of serving as an excellent feeder/support layer for the proliferation and expansion of human neural stem cells (hNSCs). In this Example, experiments were designed to address whether physical contact between the two cell types is required or whether BMSCs could support proliferation and expansion of NSCs in a contact-independent manner by way of providing soluble trophic factors to support NSC proliferation and expansion.

BMSCs, designated hBM-03-016-P1 and hBM-012-P2, were thawed, washed in BMSC medium and plated in Transwells™. Experiments in Transwells™ were performed in Costar 6-well dishes. BMSCs were plated in the Transwells™ while NSCs were placed in the bottom well. Both cell types were incubated in the same NSC medium. The use of Transwells™ enables the culturing of different cell types, for example, BMSCs and NSCs, without the two cell types being in physical contact with one another.

About 30,000 BMSCs were plated on top of the removable, porous filter of the Transwell™. After allowing the cells to adhere to the surface of the Transwell™ overnight in BMSC medium, the cells were rinsed with serum free medium and fed in complete NSC-medium (DMEM/F12+N2 Supplement+EGF+FGF). NSCs (designated THD-hWB-015-P13) were thawed, washed and plated at a density of about 40,000 cells/well on polyornithine/fibronectin coated 6 well dishes. The Transwells™ containing BMSCs were then placed in the 6 well plates containing NSCs. Controls included NSCs without BMSC in Transwells™. Sufficient media was added to both the Transwell™ and the bottom well. About 50% medium change was performed every other day. After two weeks of culture, the NSCs were harvested from each well and counted. The cells were analyzed by flow cytometry for expression of NSC markers.

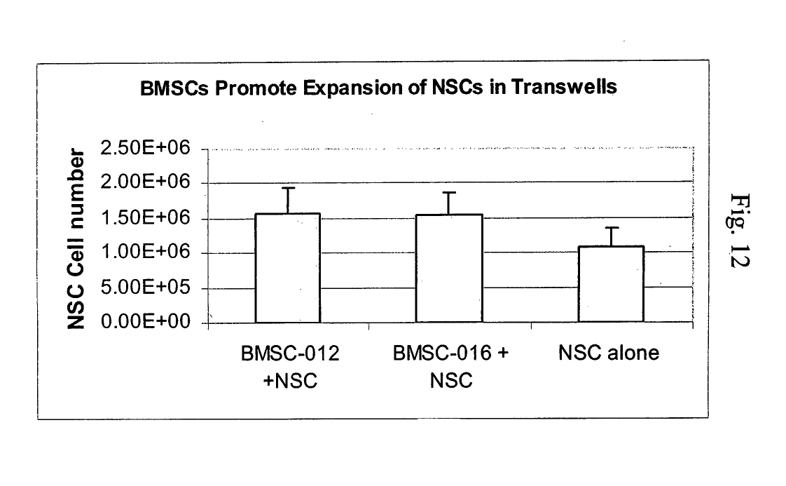

The results demonstrated that NSCs grown on coated wells together with BMSCs in Transwells™ proliferated better than NSCs cultured on coated wells without BMSCs (FIG. 12). BMSCs were observed to support growth and expansion of NSCs even in the absence of direct contact between the two cell types. Without wishing to be bound to any particular theory, it is believed that factors secreted from the BMSCs serve to supplement the NSC-medium and thereby help to support the proliferation and expansion of NSCs. FACS analysis of the NSCs grown in the presence of BMSCs in Transwells™ or grown alone in the absence of BMSC demonstrated that the NSCs were similar in phenotype. For example, the NSCs grown in both conditions were observed to be about 100% CD133 positive.

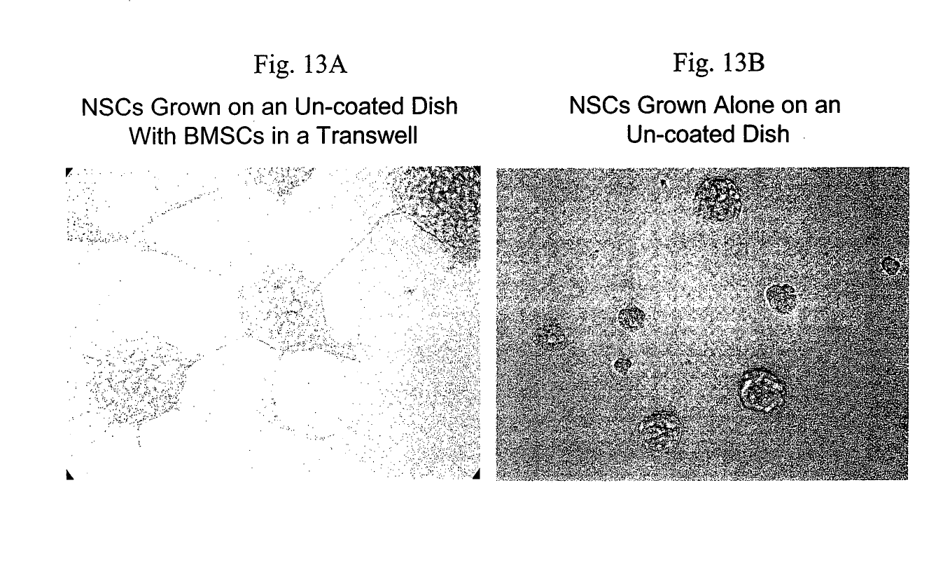

The experiments were repeated except that the 6 well dishes were not coated with polyomithine/fibronectin. About 50,000 BMSCs were plated in Transwells™. NSCs (designated THD-hWB-015-P12) were thawed and plated in either Falcon or Costar 6-well plates. BMSC grown on Transwells™ were placed in the 6-well plates and the cultures were maintained for two weeks. As controls, NSCs were cultured alone in NSC-medium with or without exogenous LIF. NSCs grown with BMSCs in Transwells™ expanded in a manner similar to NSCs grown alone in NSC-medium in the presence of exogenous LIF, but expanded significantly better than NSCs grown alone in the absence of exogenous LIF (FIG. 13). It was also observed that Costar dishes provided better results than Falcon dishes with respect to the amount of cell proliferation.

### Example 5

### Growth of NSCs in BMSC-Conditioned Medium

The results of the experiments presented in Example 4 demonstrated that BMSCs produced soluble factors that sustain NSC growth. The next set of experiments were performed with conditioned medium from cultured BMSCs to further elucidate the effects of factors secreted from BMSCs on NSC growth. The experiments disclosed herein addressed whether the secreted growth and/or other factors from BMSCs could sustain and enhance NSC growth and expansion. The experiments were designed to assess 1) whether medium conditioned by BMSCs in culture could substitute for NSC-medium supplemented with exogenous LIF for providing growth promoting effects to NSCs in culture and 2) whether medium conditioned by BMSCs in culture could provide beneficial effects to culture NSCs similar to that of culturing NSCs on polyomithine/fibronectin coated dishes.

In order to generate BMSC-CM, BMSCs were initially plated in T-80 flasks and cultured in BMSC medium. After a period of time in culture, the BMSC culture medium was replaced with NSC-medium. Therefore, BMSC-CM was obtained from BMSCs cultured with either complete NSC medium including bFGF and EGF (BMSC-CM1) or NSC medium without EGF and bFGF (BMSC-CM2). BMSCs were fed fresh NSC medium every 48 hours. At this time, the medium was removed, centrifuged to remove particulate matter and used to feed NSCs or frozen at −80° C. for assessment of cytokines. For BMSC-CM experiments, NSCs were fed with NSC growth medium comprising about 25-50% BMSC-CM that was either freshly collected from BMSCs or stored at 4° C. for as long as 2 weeks. BMSC-CM was also analyzed with Cytokine Arrays (RayBiotech Inc.) or by ELISA for presence of various cytokines.

To assess whether NSCs could be expanded in BMSC-CM, NSCs were plated on polyornithine/fibronectin coated dishes and fed every other day with a 50% change in medium with a) BMSC-CM1, b) BMSC-CM2, c) complete NSC-medium in the presence of exogenous LIF, or d) complete NSC-medium, in the absence of exogenous LIF. For the initial two changes in BMSC-CM1 and BMSC-CM2, fresh EGF and FGF was added to each BMSC-CM, but the addition of EGF and FGF to the BMSC-CM was later stopped.

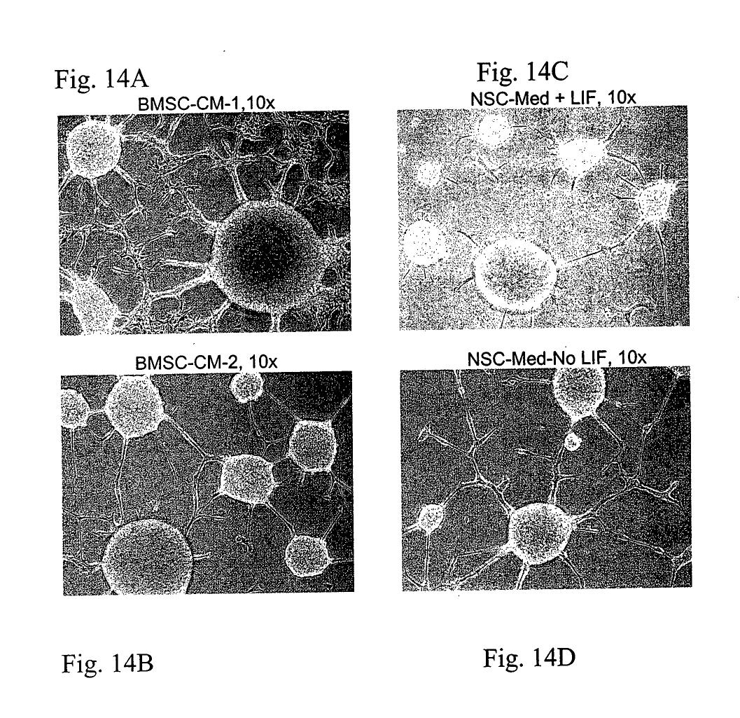

The results demonstrate that NSCs grew equally well in BMSC-CM or in complete NSC-medium. FIG. 14 depicts representative fields of the NSCs cultured under the different conditions. In the presence of BMSC-CM1, it was observed that NSCs formed large adherent neurospheres, with the neurospheres having cells growing out of the neurospheres. It was also observed that some of the NSCs cultured in the presence of BMSC-CM1 formed a monolayer. In the case where NSCs were cultured in BMSC-CM2, it was also observed that neurospheres formed from the NSCs. However, fewer large neurospheres formed and fewer monolayers appeared as compared with NSCs cultured in the presence of BMSC-CM1. In the presence of complete NSC-medium, NSCs grew as several neurospheres connected to each other and exhibited outgrowing cells from the neurospheres. In any event, cells in each culture set were harvested after 10 days in culture and counted for the number of cells to compare the effects of the different culture conditions on the proliferation of NSCs. The number of cells harvested from BMSC-CM1 (approximately 3.4e6 cells) was similar to that of complete NSC-medium in the presence of exogenous LIF (approximately 3.3e6). The number of cells obtained in BMSC-CM2 was approximately 2.85e6.

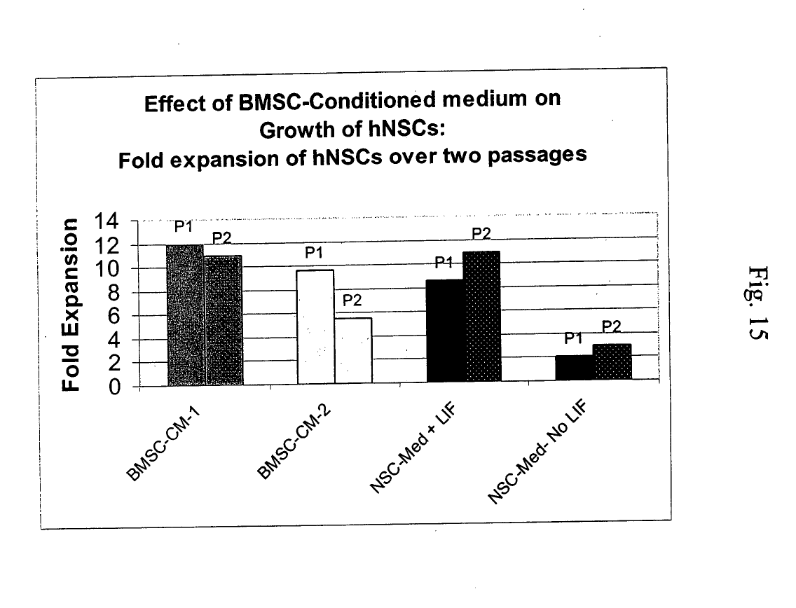

The next set of experiments tested whether NSCs could be expanded in BMSC-CM even on uncoated dishes. The four groups of NSC are as follows: 1) NSCs cultured in BMSC-CM1; 2) NSCs cultured in BMSC-CM2; 3) NSCs cultured in complete NSC-medium in the presence of exogenous LIF; and 4) NSCs cultured in complete NSC-medium in the absence of exogenous LIF. The cells were cultured for 2 weeks, passaged by trypsinization and treated with same conditions for another 2 weeks. The greatest expansion of NSCs was observed in BMSC-CM1 followed by BMSC-CM2 and then NSC-medium+LIF for the first 2 weeks (bars represented by P1, FIG. 15). After passage, BMSC-CM1 produced similar expansion of NSCs as NSC medium+LIF (represented by P2 in FIG. 15). Cells treated with BMSC-CM2 continued to proliferate and did better than cells treated with NSC medium-without LIF. Thus, the present data indicates that BMSC-CM is a good source of growth factors for hNSCs and is able to induce proliferation of NSCs at rates equal to or higher than that of NSC-medium supplemented with exogenous LIF. In addition, NSCs cultured with BMSC-CM can be passaged by trypsinization and further expanded to increase cell numbers.

Cytokine Array Analysis of BMSC-CM

BMSCs were cultured in complete NSC-medium. Medium was collected at 48 hours and the cytokine profile was determined using cytokine assays. RayBio Human Cytokine Antibody Array C Series 1000 (combination of Arrays VI and VII) was purchased from RayBiotech, Inc (Norcross, Ga.). The array membranes were treated with the samples as described in the manufacturer's instruction manual. The final detection of the cytokines was performed using the ECL-Plus system (Amersham, Piscataway, N.J.). The signals were visualized on X-ray film (Amersham, Piscataway, N.J.).

The results from the cytokine profile demonstrated that various cytokines/factors were present in the BMSC-CM based on the intensity of the signal over and above the negative controls on the same array. The cytokines/factors include, but are not limited to LIF, brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

Without wishing to be bound by any particular theory, BMSC-CM comprises several factors which includes factors already present in the culture medium including EGF and bFGF in addition to factors that are either secreted by BMSCs or that were induced by the medium. The factors listed herein are based on the assessment of signals above negative controls on arrays for 120 cytokines that were present on the tested arrays.

Some of the cytokines that were detected above background level included, but are not limited to EGF and bFGF. Other clearly expressed cytokines were HGF (hepatocyte growth factor), M-CSF (macrophage colony-stimulating factor) and TIMP-1 and TIMP-2 (tissue inhibitors of metalloproteinase 1 and 2, respectively). Neurotrophic factors included, but are not limited to BDNF, NT3, GDNF and CNTF. IGFBPs, and FGF-6 were present. Many chemokines, proinflammatory cytokines and angiogenic factors, for example, VEGF were also produced. LIF was also produced by BMSCs which was also confirmed with ELISA. In the ELISA assay, the actual amount of each tested secreted factor from the BMSCs was assessed by subtracting the control medium levels from the levels observed in BMSC-CM.

From the disclosure presented herein, it can be concluded that BMSCs produce soluble factors that promote NSC growth. Direct contact of NSCs with BMSC is not necessary, as BMSC conditioned can also sustain the growth and expansion of NSCs. The present disclosure shows that contact dependent co-culture produces the highest expansion of NSCs suggesting a synergistic effect of physical contact with BMSCs or BMSC-products and soluble factors produced by BMSCs with NSCs for the expansion of NSCs.

### Example 6

### NSCs Cultured on BMSCs or in BMSC-CM Exhibited Reduced Expression Levels of MHC Molecules but Retained the Same Phenotypic Profile of NSCs Grown Alone in Standard Conditions

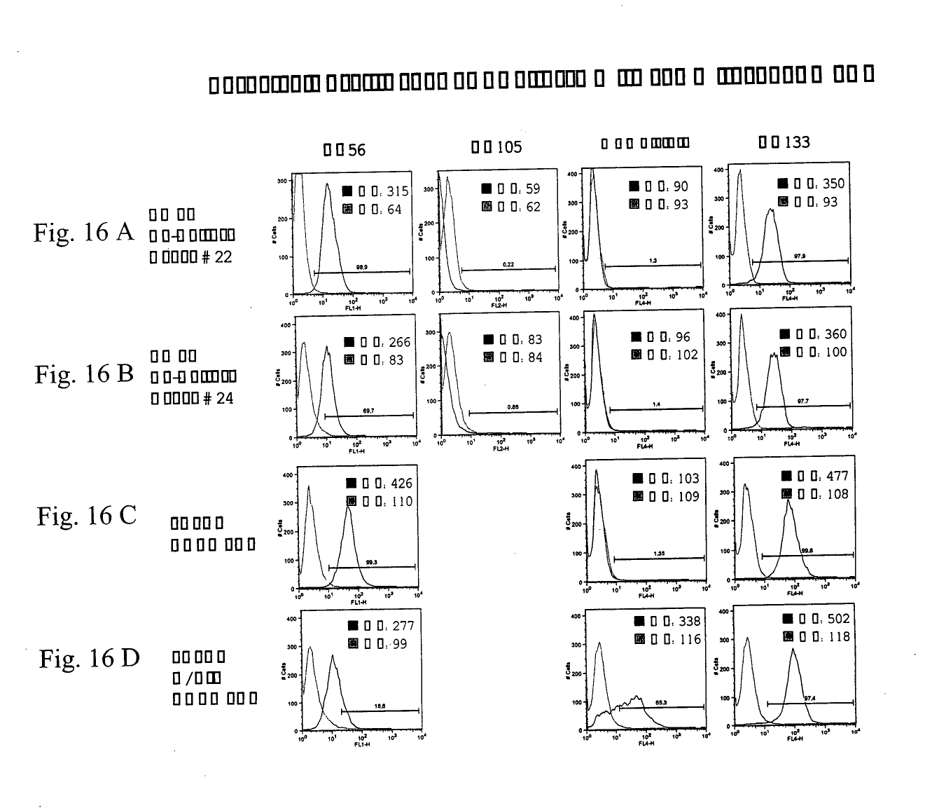

NSCs expanded in Example 3 were analyzed by Flow Cytometry for expression of various BMSC and NSC markers. NSCs co-cultured with BMSC retained the same phenotypic profile (e.g. positive for CD56 and CD133 and negative for CD105) as NSCs cultured without BMSCs in NSC growth medium supplemented with exogenous LIF, with the exception that NSC co-cultured with BMSC exhibited no detectable expression of MHC Class II molecules (FIG. 16). It was observed that MHC Class II molecules were induced on NSCs when exogenous LIF was added to NSC growth medium. In contrast, NSCs co-cultured with BMSCs were not observed to express MHC Class II molecules above baseline which may serve to be advantageous for transplantation. Further, NSCs isolated from co-cultures did not express the BMSC marker CD105 above baseline demonstrating an efficient depletion of BMSCs from the co-cultures.

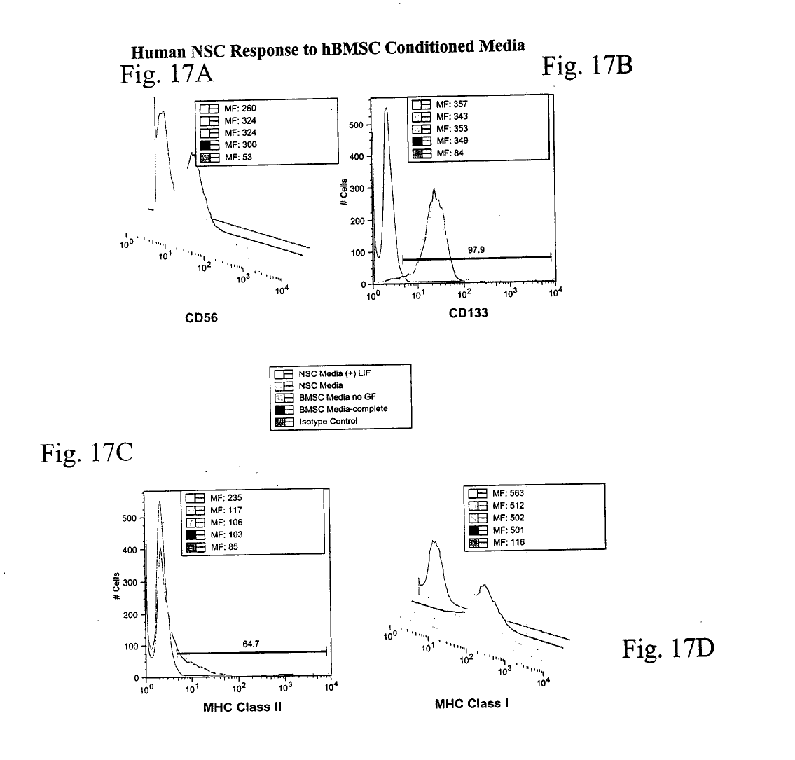

Similarly NSCs cultured with BMSC-CM also retained the same phenotypic profile of NSCs cultured with standard NSC medium+LIF except that they showed baseline levels of MHC Class II and reduced MHC Class I (FIG. 17). Four groups of NSC were subjected to FACS analysis for the expression of CD133, MHC Class II molecules, MHC Class I molecules, and CD56 (FIG. 17). The results confirmed previous observations that NSCs grown in the presence of exogenous LIF expressed MHC Class II molecules and exhibited an increased median fluorescence of MHC Class I molecules. However NSCs grown in BMSC-CM were not observed to express MHC Class II molecules and had lower levels of MHC Class I molecule expression. The expression of CD133 was similar in all four cases. All the cells tested expressed CD56 although cells cultured in the presence of exogenous LIF exhibited a reduced median fluorescence of CD56.

### Example 7

### Regulation of MHC Class I and Class II Expression on NSCs by Expanding on BMSCs

Additional experiments were performed to assess the expression of both MHC Class I and Class II molecules by co-cultured NSCs. NSCs were isolated from fetal brain and cultured for 13 passages in NSC medium (THD-WB-015, P13). The NSCs were then cultured in the following 4 conditions for 12 days with medium change every other day:
1. 62,500 NSCs alone in a coated T25 flask with complete NSC medium
2. 62,500 NSCs alone in a coated T25 flask with complete NSC medium+LIF
3. 62,500 NSCs on top of 250,000 BMSCs (Donor-012) feeder layer in complete NSC medium
4. NSCs on top of 250,000 BMSCs (Donor-016) feeder layer in complete NSC medium

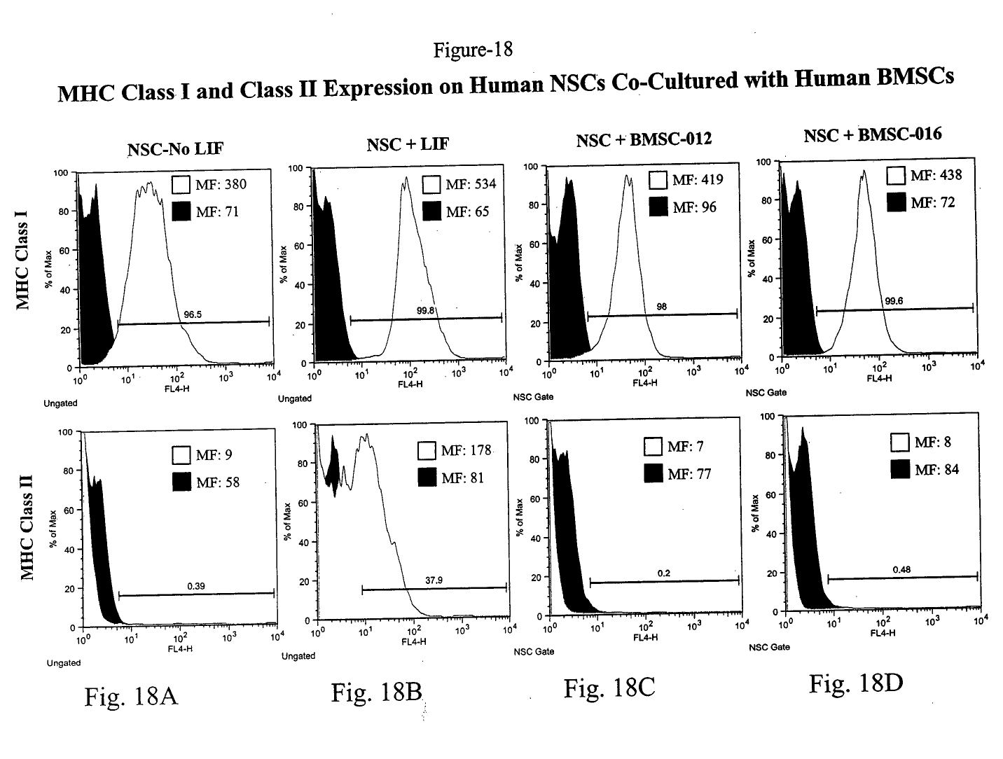

After 12 days the cells were harvested by trypsinization. The cells were counted and analyzed by flow cytometry for expression of CD133, CD105, MHC Class I and MHC Class II. In the case of the co-cultures, all cells were acquired and the NSCs were gated based on their light scatter and CD133 expression. FIG. 18 shows the expression of MHC Class I and Class II molecules by the NSCs cultured under the various conditions (Black=isotype control; Gray=Class I or Class II).

The results show that all NSCs expressed MHC Class I. However when NSCs were cultured in the presence of exogenous LIF, there was a significant increase in the MHC class I expression on the cells, as reflected by the increase in Log Median Fluorescence Intensity when compared with NSCs cultured in the absence of exogenous LIF. Co-cultured NSCs expressed significantly lower levels of MHC Class I when compared to NSCs expanded with LIF (Table 4).

TABLE 4

| | Log MF MHC Class I- | |
|---|---|---|
| Treatment | isotype control | Fold Cell Expansion |
| NSC alone No LIF | 209 | 8.16 X |
| NSC alone + LIF | 469 | 13.5 X |
| NSC + BMSC-012 | 323 | 16 X |
| NSC + BMSC-016 | 366 | 26.7 X |

NSCs did not express MHC Class II molecules constitutively. However, LIF induced the expression of MHC Class II molecules on 38% of the cells. NSCs cultured with BMSCs were similar to NSCs cultured in the absence of LIF and did not express MHC Class II above baseline.

The results demonstrate that an advantage of expanding NSCs on BMSCs is to reduce and/or prevent expression of immuno-regulatory MHC Class I and II molecules on NSCs which makes them more suitable for clinical transplantation.

The disclosures of each and every patent, patent application, and publication cited herein are hereby incorporated herein by reference in their entirety.

It will be apparent to those skilled in the art that various modifications and variations can be made in the methods and compositions of the present invention without departing from the spirit or scope of the invention. Thus, it is intended that the present invention cover the modifications and variations of the present invention provided they come within the scope of the appended claims and their equivalents.

1. A composition comprising: (a) an isolated Bone Marrow Stromal Cell (BMSC); and (b) a chemically defined culture medium comprising Neural Stem Cell (NSC) growth medium and factors secreted by said BMSC.

2. The composition of claim 1, wherein said culture medium does not contain exogenous Leukemia Inhibitory Factor (LIF).

3. The composition of claim 1, wherein said factors are selected from the group consisting of growth factors, trophic factors and cytokines.

4. The composition of claim 1, wherein said factors are selected from the group consisting of LIF, brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-Ira, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

5. The composition of claim 1, further comprising an isolated NSC.

6. The composition of claim 5, wherein said NSC is physically contacted with said BMSC.

7. The composition of claim 5, wherein said NSC is not physically contacted with said BMSC.

8. The composition of claim 5, wherein said NSC is derived from the central nervous system of a human.

9. The composition of claim 1, wherein said BMSC is derived from a human.

10. The composition of claim 5, wherein exogenous genetic material has been introduced into said NSC.

11. The composition of claim 1, wherein exogenous genetic material has been introduced into said BMSC.

12. A Bone Marrow Stromal Cell conditioned medium (BMSC-CM) comprising a chemically defined culture medium comprising Neural Stem Cell (NSC) growth medium and factors secreted by an isolated BMSC.

13. The BMSC-CM of claim 12, wherein said BMSC-CM does not contain exogenous LIF.

14. The BMSC-CM of claim 12, wherein said factors are selected from the group consisting of growth factors, trophic factors and cytokines.

15. The BMSC-CM of claim 12, wherein said factors are selected from the group consisting of LIF, brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

16. A method of modulating Major Histocompatibility Complex (MHC) molecule expression on an isolated NSC, said method comprising co-culturing cells comprising an isolated BMSC and an isolated NSC.

17. The method of claim 16, wherein said cells are co-cultured in the absence of exogenous LIF.

18. The method of claim 16, wherein said NSC is physically contacted with said BMSC.

19. The method of claim 16, wherein said NSC is not physically contacted with said BMSC.

20. The method of claim 16, wherein said NSC is derived from the central nervous system of a human.

21. The method of claim 16, wherein said BMSC is derived from a human.

22. The method of claim 16, wherein exogenous genetic material has been introduced into said NSC.

23. The method of claim 16, wherein exogenous genetic material has been introduced into said BMSC.

24. A method of modulating MHC molecule expression on an isolated NSC, said method comprising culturing said NSC with Bone Marrow Stromal Cell conditioned medium (BMSC-CM), wherein said BMSC-CM comprises NSC growth medium and factors secreted by said BMSC.

25. The method of claim 24, wherein said BMSC-CM does not contain exogenous LIF.

26. The method of claim 24, wherein said BMSC-CM is essentially free of BMSCs.

27. The method of claim 24, wherein said factors are selected from the group consisting of growth factors, trophic factors and cytokines.

28. The method of claim 24, wherein said factors are selected from the group consisting of LIF, brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

29. The method of claim 24, wherein said NSC is derived from the central nervous system of a human.

30. The method of claim 24, wherein exogenous genetic material has been introduced into said NSC.

31. An isolated NSC prepared from the method of co-culturing an isolated BMSCs with an isolated NSC.

32. The isolated NSC of claim 31, wherein said NSC exhibits a reduced expression of MHC Class I molecule.

33. The isolated NSC of claim 31, wherein said NSC exhibits a baseline level of MHC Class II molecule.

34. The isolated NSC of claim 31, wherein said NSC is derived from the central nervous system of a human.

35. The isolated NSC of claim 31, wherein exogenous genetic material has been introduced into said NSC.

36. An isolated NSC prepared by culturing an isolated NSC in BMSC-CM, wherein said BMSC-CM comprises a chemically defined culture medium comprising NSC growth medium and factors secreted by an isolated BMSC.

37. The isolated NSC of claim 36, wherein said NSC exhibits a reduced expression of MHC Class I molecule.

38. The isolated NSC of claim 36, wherein said NSC exhibits a baseline level of MHC Class II molecule.

39. The isolated NSC of claim 36, wherein said NSC is derived from the central nervous system of a human.

40. The isolated NSC of claim 36, wherein exogenous genetic material has been introduced into said NSC.

41. A neural cell culture device comprising:
(a) an isolated NSC;
(b) an isolated BMSC;
(c) a NSC growth medium, wherein said NSC growth medium comprises factors secreted from said isolated BMSC; and
(d) a means for keeping said NSC and said BMSC from coming into physical contact with one another.

42. The device of claim 41 further comprising a filter or membrane which keeps said NSC and said BMSC from coming into physical contact with one another.

43. The device of claim 42 wherein said filter or membrane has pores to allow factors secreted from said BMSC to cross said filter or membrane.
